(19) United States
(12) Patent Application Publication
(10) Pub. No.: US 2011/0065115 A1
(43) Pub. Date: Mar. 17, 2011

(54) METHODS FOR IDENTIFYING AN INCREASED LIKELIHOOD OF RECURRENCE OF BREAST CANCER

(75) Inventors: James L. Wittliff, Louisville, KY (US); Sarah A. Andres, Floyds Knobs, IN (US)

(73) Assignee: University of Louisville Research Foundation, Inc., Louisville, KY (US)

(21) Appl. No.: 12/885,720

(22) Filed: Sep. 20, 2010

Related U.S. Application Data
(63) Continuation of application No. 12/630,212, filed on Dec. 3, 2009, now abandoned.
Application No. 12/630,212 is a continuation of application No. PCT/US2008/006963, filed on Jun. 3, 2008.
(60) Provisional application No. 60/933,091, filed on Jun. 4, 2007.

(51) Int. Cl. C12Q 1/68

(52) U.S. Cl. 435/6

(57) ABSTRACT

Methods of identifying a mammal having an increased likelihood of recurrence of breast cancer includes identifying in a breast tissue sample of the mammal expression of at least two genes selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3) and subsets of the genes.

### RELATED APPLICATION

This application is a continuation of U.S. application Ser. No. 12/630,212, filed Dec. 3, 2009, which is a continuation of International Application No. PCT/US2008/006963, which designates the United States and was filed on Jun. 3, 2008, published in English, which claims the benefit of U.S. Provisional Application No. 60/933,091, filed Jun. 4, 2007. The entire teachings of the above application(s) are incorporated herein by reference.

### BACKGROUND OF THE INVENTION

Breast cancer is a major health concern and one of the most prevalent forms of cancer in woman. Breast cancer has the second highest mortality rate of cancers and about 15% of cancer-related deaths in women are do to breast cancer (SEER Cancer Statistics Review 1975-2005, NCI, Ries, L. A. G., et al., (eds) (2008)). It has been estimated that about 13% of women born in the United. States will be diagnosed with breast cancer in their lifetime (SEER Cancer Statistics Review 1975-2005, NCI, Ries, L. A. G., et al., (eds) (2008)). Currently, techniques to diagnosis, in particular, to identify women at an increased likelihood of recurrence of breast cancer, methods of treating breast cancer and methods to monitor progress of treatment regimens for breast cancer include the presence of certain tumor markers in breast tissue biopsies. However, such techniques may be inaccurate in detecting breast cancer and assessing therapy options. Thus, there is a need to develop new, improved and effective methods of identifying a woman having an increased likelihood of recurrence of breast cancer, which may determine a course of therapy selection and prognosis.

### SUMMARY OF THE INVENTION

The present invention relates to methods of identifying a mammal having an increased likelihood of recurrence of breast cancer.

In an embodiment, the invention is a method for identifying a mammal having an increased likelihood of recurrence of breast cancer, comprising the step of identifying in a breast tissue sample of the mammal expression of at least two genes, wherein the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

The methods of the invention can be employed to identify a mammal at a heightened risk for recurrence of breast cancer. Advantages of the claimed invention include, for example, improved accuracy of methods to identify mammals that have an increased likelihood of recurrence of breast cancer, which can be of value in the determination of treatment regimens and prognosis. The claimed methods can be employed to assist in the prevention and treatment of breast cancer and, therefore, avoid serious illness and death consequent to breast cancer.

### BRIEF DESCRIPTION OF THE FIGURES

FIG. 1 depicts procedures employed in identifying genes for use in the methods.

FIGS. 2A, 2B, 2C and 2D depict laser capture microdissection (LCM) breast cancer cells. FIG. 2B is before LCM and FIG. 2C is after LCM. FIG. 2A is 10× magnification. FIGS. 2B, 2C and 2D are 20× magnification.

FIGS. 3A, 3B, 3C and 3D depict laser capture microdissection (LCM) breast cancer stromal cells. FIG. 3B is before LCM and FIG. 3C is after LCM. FIG. 3A is 10× magnification. FIGS. 3B, 3C and 3D are 20× magnification.

FIG. 4 depicts representative gene expression in 14 genes when tissue specimens were processed concurrently. (Mean±SD shown).

FIGS. 5A, 5B, 5C, 5D, 5E and 5F depict representative Kaplan-Meier plots of the EVL and IL6 genes depicting disease-free survival (FIGS. 5A and 5B), overall survival (FIGS. 5C and 5D) and event-free survival (FIGS. 5E and 5F).

FIGS. 6A and 6B depict representative expression of 14 genes (Table 2) when tissue specimens are processed concurrently. (Mean±SD shown).

FIGS. 7A and 7B depict representative gene expression results (Mean±SD shown) with tissue specimens processed independently for genes listed in Table 2. Comparison of variation between tissue sections is depicted in FIG. 7A and comparison of qPCR runs is depicted in FIG. 7B.

FIGS. 8A, 8B and 8C depict scatter plots of representative expression distribution of the NAT1, ESR1 and GABRP genes in 78 intact tissue sections.

FIGS. 9A, 9B, 9C and 9D depict representative comparisons of gene expression between intact tissue sections and LCM-procured cells. FIGS. 9A and 9B depict expression of the NAT1 and ESR1 genes that do not show a statistical difference in expression from an intact tissue section compared to LCM procured cells. FIGS. 9C and 9D depict expression of the PFKP and PLK1 genes where there is a statistical difference in expression from an intact tissue section compared to LCM procured cells.

FIGS. 10A, 10B, 10C, 10D, 10E and 10F depict scatter plots of representative correlations between gene expression analyzed by qPCR and microarray. FIGS. 10A, 10B and 10C depict expression of the ESR1, NAT1 and SCUBE2 genes, which had the best correlation. FIGS. 10D, 10E and 10F depict expression of the MAPRE2, PLK1 and GMPS genes, which had the worst correlation.

FIGS. 11A and 11B depict scatter plots of comparisons between gene expression of estrogen receptor (FIG. 11A) and progestin receptor (FIG. 11B) in 97 patient specimens. One outlier sample was removed during analysis of the progestin receptor.

FIG. 12 depicts the likelihood of death from breast cancer based on various patient characteristics.

FIGS. 13A, 13B, 13C, 13D, 13E, 13F, 13G, 13H and 13I depict Kaplan-Meier plots showing disease-free survival (FIGS. 13A, 13 B3 and 13C), overall survival (FIGS. 13D, 13E and 13F) and event-free survival (FIGS. 13G, 13H and 13I) of known prognostic factors.

FIGS. 14A, 14B, 14C, 14D, 14E, 14F, 14G, 14H and 14I depict representative Kaplan-Meier plots of expression of the SLC43A3, GABRP and DSC2 genes showing the most statistical significance. Disease free survival is depicted in FIGS. 14A, 14B and 14C. Overall survival is depicted in FIGS. 14D, 14E and 14F. Event free survival is depicted in FIGS. 14G, 14H and 14I.

FIGS. 15A, 15B, 15C and 15D depict Kaplan-Meier analyses of the ESR1 and GABRP genes using predetermined cut-offs of 2 relative gene units (ESR1) and 64 relative gene units (GABRP). Disease-free survival is depicted in FIGS. 15A and 15B and overall survival is depicted in FIGS. 15C and 15D.

FIGS. 16A and 16B depict Kaplan-Meier analysis of Model 1 (See Table 10) developed through PARTEK®GENOMICS SUITE™ (PARTEK Incorporated, St. Louis, Mo.) for predicting disease recurrence. Disease-free survival is depicted in FIG. 16A and overall survival is depicted in FIG. 16B.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The invention generally is directed to methods for identifying a mammal having an increased likelihood of recurrence of breast cancer by identifying in a breast tissue sample the expression of particular genes.

An embodiment of the invention is a method for identifying a mammal having an increased likelihood of recurrence of breast cancer, comprising the step of identifying in a breast tissue sample of the mammal expression of at least two genes, wherein the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3). The genes identified are listed in Table 1, which includes UniGene identifies (Hs), a description of the gene and an mRNA Accession Number that corresponds to the mRNA of the gene listed. The TBC1D9 gene is also referred to as the “KIAA0882 gene.” The ST8SIA1 gene is also referred to as the “SIAT8A gene.”

“An increased likelihood of recurrence of breast cancer,” as used herein, means that the mammal had at least one incident of a diagnosis of breast cancer and has an elevated probability of having the breast cancer return. The mammal, for example a human patient, may have undergone at least one member selected from the group consisting of a surgical treatment for breast cancer, a chemotherapy treatment for breast cancer and a radiation treatment for breast cancer. An increased likelihood of breast cancer recurrence in a human can be consequent to several factors including, for example, the nodal status, estrogen and progesterone receptor levels, grade of cancer and stage of the previous breast cancer or cancers.

For example, in a meta-analysis (from seven different studies) of more than about 3,500 patients who had received some type of post-surgical adjuvant therapy for breast cancer, risk of cancer recurrence was greatest during the first two years following surgery. After this period, the research showed a steady decrease in the risk of recurrence until year five when the risk of recurrence declined slowly and averaged about 4.3% per year (Saphner T, et al., J Clin Oncol. 14:2738-2746 (1996)). Some proportion of breast cancer recurrences seen in this study occurred more than about five years after surgery, between about six to about 12 years after surgery, even in patients who typically would be considered at low risk for recurrence because their cancer had not spread to the lymph nodes at the time of diagnosis (node-negative). This study shows that through at least about 12 years of follow-up, the risk of breast cancer recurrence remains appreciable and even some patients considered low risk have some risk of the cancer coming back.

In another meta-analysis, of about 37,000 women with early breast cancer, conducted by the Early Breast Cancer Trialists' Collaborative Group, it was found that through the first about 10 years after diagnosis, the cumulative incidence of recurrence and breast cancer-related deaths continued to increase, with a substantial portion of recurrences and breast-cancer related deaths occurring beyond about five years after diagnosis. The recurrence rate among patients who did not receive adjuvant hormonal therapy was about 50% in node-positive patients and about 32.4% in node-negative patients throughout the first 10 years after diagnosis (Early Breast Cancer Trialists' Collaborative Group. Tamoxifen for early breast cancer: an overview of the randomized trials. Lancet 351:1451-1466 (1998)). These data showed that some years of adjuvant Tamoxifen treatment substantially improved the 10-year survival of women with estrogen receptor-positive tumors and of women whose tumors are of unknown ER status, even in women who had node-negative disease (Fisher B, et al., N Engl J Med. 320:479-484 (1989); Fisher B, et al., Lancet 364:858-868 (2004)). Thus, an increased likelihood of recurrence of breast cancer can be, for example, depending on the treatment of the previous breast cancer, the nodal status, the estrogen and progesterone receptor levels, the grade of cancer and the stage of the previous cancer, about a 30%, about a 35%, about a 40%, about a 45%, about a 50%, about a 55%, about a 60%, about a 65%, about 70%, about a 75%, about a 80%, about a 85%, about a 90%, about a 95% or about a 100% increase in return of breast cancer compared to an average return of breast cancer.

In an embodiment, the methods of the invention can include identifying a mammal having an increased likelihood of recurrence of breast cancer by identifying genes in the breast tissue sample that consist of genes listed in Tables 1-36. In another embodiment, the methods of the invention can include identifying a mammal having an increased likelihood of recurrence of breast cancer by identifying genes selected from the group consisting of genes listed in Tables 1-36.

Breast tumors can be either benign or malignant. Benign tumors are not cancerous, generally do not spread to non-breast tissues and are not life threatening. Benign tumors can generally be removed and do not recur. Malignant tumors are cancerous and can form metastases to non-breast tissues and organs by entering the systemic circulatory system (arteries, veins) or lymphatic circulatory system. The methods described herein can be employed to identify a mammal at an increased risk of recurrence of a malignant breast tumor.

In another embodiment, the expressed genes identified in the breast tissue sample consist of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

In an additional embodiment, the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1) and Hs.95612 (DSC2).

In a further embodiment, the expressed genes identified in the breast tissue sample consist of Hs.125867 (EVL), Hs.591847 (NAT1), Hs.208124 (ESR1), Hs.26225(GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136(SLC39A6), Hs.82128 (TPBG), Hs.95243 (TCEAL1) and Hs.95612 (DSC2).

In yet another embodiment, the genes are selected from the group consisting of Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

In still another embodiment, the expressed genes identified in the breast tissue sample consist of Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

In an additional embodiment, the genes are selected from the group consisting of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

In yet another embodiment, the expressed genes identified in the breast tissue sample consist of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

In still another embodiment, the genes are selected from the group consisting of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1) and Hs.95612 (DSC2).

In another embodiment, the expressed genes identified in the breast tissue sample consist of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1) and Hs.95612 (DSC2).

In still another embodiment, the genes are selected from the group consisting of Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

In a further embodiment, the expressed genes identified in the breast tissue sample consist of Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

In yet another embodiment, the genes are selected from the group consisting of Hs.79136 (SLC39A6), Hs.82128 (TPBG), Hs.480819 (TBCID9), Hs.592121 (RABEP1) and Hs.532082 (IL6ST).

In an additional embodiment, the expressed genes identified in the breast tissue sample consist of Hs.79136 (SLC39A6), Hs.82128 (TPBG), Hs.480819 (TBCID9), Hs.592121 (RABEP1) and Hs.532082 (IL6ST) is identified in the breast tissue sample.

In a further embodiment, the genes are selected from the group consisting of Hs.79136 (SLC39A6), Hs.82128 (TPBG), Hs.480819 (TBCID9) and Hs.592121 (RABEP1).

In still another embodiment, expression of Hs.79136 (SLC39A6), Hs.82128 (TPBG), Hs.480819 (TBC1D9) and Hs.592121 (RABEP1) is identified in the breast tissue sample.

In still another embodiment, the genes are selected from the group consisting of Hs.79136 (SLC39A6), Hs.82128 (TPBG) and Hs.480819 (TBC1D9).

In a further embodiment, expression of Hs.79136 (SLC39A6), Hs.82128 (TPBG) and Hs.480819 (TBC1D9) is identified in the breast tissue sample.

In an additional embodiment, the genes are selected from the group consisting of Hs.26225 (GABRP), Hs.523468 (SCUBE2), Hs.592121 (RABEP1), Hs.95612 (DSC2), Hs.1594 (CENPA), Hs.524134 (GATA3), Hs.532824 (MAPRE2), and Hs.99962 (SLC43A3).

In yet another embodiment, the expressed genes identified in the breast tissue sample consist of Hs.26225 (GABRP), Hs.523468 (SCUBE2), Hs.592121 (RABEP1), Hs.95612 (DSC2), Hs.1594 (CENPA), Hs.524134 (GATA3), Hs.532824 (MAPRE2) and Hs.99962 (SLC43A3) is identified in the breast tissue sample.

In an additional embodiment, the genes are selected from the group consisting of Hs.208124 (ESR1), Hs.591847 (NAT1) and Hs.523468 (SCUBE2).

In another embodiment, the expressed genes identified in the breast tissue sample consist of Hs.208124 (ESR1), Hs.591847 (NAT1) and Hs.523468 (SCUBE2) is identified in the breast tissue sample.

In yet another embodiment, one of the genes is Hs.99962 (SLC43A3).

In yet another embodiment, the genes are selected from group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.523468 (SCUBE2), Hs.592121 (RABEP1), Hs.79136 (SLC39A6), Hs.82128 (TPBG), Hs.95243 (TCEAL1), Hs.654961 (FUT8), Hs.184339 (MELK), Hs.26010 (PFKP), Hs.437638 (XBP1), Hs.470477 (PTP4A2), Hs.473583 (YBX1), Hs.480938 (LRBA), Hs.524134 (GATA3), Hs.531668 (CX3CLI) and Hs.99962 (SLC43A3), which can be associated with estrogen-receptor status (estrogen-receptor positive breast tissue sample, estrogen-receptor negative breast tissue sample) the breast tissue sample.

In another embodiment, the genes are identified in an estrogen-receptor positive breast tissue sample. “Estrogen-receptor positive breast tissue sample,” as used herein, means that the levels of estrogen receptor protein measured are greater than about 10 fmol/mg protein (e.g., about 15 fmol/mg protein) as measured by established techniques, which include at least one member selected from the group consisting of radioligand binding, Enzyme Immuno Assay and semi-quantitative immunohistochemical assay (see, for example, Wittliff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W.B. Saunders Co. (1998)).

The genes identified in estrogen-receptor positive a breast tissue samples can include at least one of the genes selected from the group consisting of Hs.125867(EVL), Hs.591847 (NAT1), Hs.208124 (ESR1), Hs.480819 (TBC1D9), Hs.523468 (SCUBE2), Hs.592121 (RABEP1), Hs.79136 (SLC39A6), Hs.95243 (TCEAL1), Hs.654961 (FUT8) and Hs.531668 (CX3CL1). In an embodiment, the genes identified include Hs.208124 (ESR1) and at least one member selected from the group consisting of Hs.125867(EVL), Hs.591847 (NAT1), Hs.208124 (ESR1), Hs.480819 (TBC1D9), Hs.523468 (SCUBE2), Hs.592121 (RABEP1), Hs.79136 (SLC39A6), Hs.95243 (TCEAL1), Hs.654961 (FUT8) and Hs.531668 (CX3CL1).

In another embodiment, the genes are identified in an estrogen-receptor negative breast tissue sample. “Estrogen-receptor negative breast tissue sample,” as used herein, means that the levels of estrogen receptor protein measured are less than about 10 finol/mg protein (e.g., about 15 fmol/mg protein) as measured by established techniques, which include at least one member selected from the group consisting of radioligand binding, Enzyme ImmunoAssay and semi-quantitative immunohistothernical assay (see, for example, Wittliff, J. L. et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W. B, Saunders Co. (1998)).

The genes identified in an estrogen-receptor negative breast tissue sample can include at least one of the genes selected from the group consisting of Hs.26225 (GABRP), Hs.408614 (ST8SIA1), Hs.184339 (MELK) and Hs.437638 (XBP1).

In yet another embodiment, the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.592121 (RABEP1), Hs.79136 (SLC39A6), Hs.95243 (TCEAL1), Hs.654961 (FUT8), Hs.184339 (MELK), Hs.26010 (PFKP), Hs.437638 (XBP1), Hs.470477 (PTP4A2), Hs.524134 (GATA3), Hs.531668 (CX3CL1) and Hs.99962 (SLC43A3), which can be associated with progestin receptor status (progestin-receptor positive breast tissue sample, progestin-receptor negative breast tissue sample) the breast tissue sample.

The genes are identified can be from a progestin-receptor positive breast tissue sample.

“Progestin-receptor positive breast tissue sample,” as used herein, means that the levels of progestin receptor protein measured are greater than about 10 fmol/mg protein (e.g., about 15 fmol/mg protein) as measured by established techniques, which include at least one member selected from the group consisting of radioligand binding, Enzyme ImmunoAssay and semi-quantitative immunohistochemical assay (see, for example, Witttiff, J. L., et al., Steroid and Peptide Hormone Receptors: Methods, Quality Control and Clinical Use. In: K. L Bland and E. M. Copeland III (eds.), The Breast: Comprehensive Management of Benign and Malignant Diseases, Chapter 25, pp. 458-498, Philadelphia, Pa.: W.B. Saunders Co. (1998)).

The genes identified in a progestin-receptor positive breast tissue sample include at least one of the genes selected from the group consisting of Hs.125867 (EVL), Hs.591847 (NAT1), Hs.208124 (ESR1), Hs.480819 (TBC1D9). Hs.592121 (RABEP1), Hs.79136 (SLC39A6), Hs.654961 (FUT8), Hs.437638 (XBP1) and Hs.470477 (PTP4A2).

The genes can be identified in a progestin-receptor negative breast tissue sample.

“Progestin-receptor negative breast tissue sample,” as used herein, means that the levels of progestin receptor protein measured are less than about 10 fmol/mg protein (e.g., about 15 fmol/mg protein) as measured by established techniques, which include at least one member selected from the group consisting of radioligand binding, Enzyme ImmunoAssay and semi-quantitative immunohistochemical assay (see, for example, Wittliff, J. L., et al., Steroid and Peptide Hormone Receptors: Methods, Quality Control and Clinical Use. In: K. I. Bland and E. M. Copeland III (eds.), The Breast: Comprehensive Management of Benign and Malignant Diseases, Chapter 25, pp. 458-498, Philadelphia, Pa.: W.B. Saunders Co. (1998)).

The genes identified in a progestin-receptor negative breast tissue sample can include at least one of the genes selected from the group consisting of Hs.26225 (GABRP), Hs.408614 (ST8SIA1) and Hs.184339 (MELK).

In another embodiment, the genes are selected from the group consisting of Hs.208124 (ESR1), Hs.26225 (GABRP), Hs.504115 (TRIM29), Hs.1594 (CENPA), Hs.184339 (MELK), Hs.592049 (PLK1), Hs.370834 (ATAD2), Hs.470477 (PTP4A2), Hs.473583 (YBX1) and Hs.83758 (CKS2), which can be associated with menopausal status of the mammal (e.g., peri-menopausal, pre-menopausal, post-menopausal).

The genes selected from the group consisting of Hs.208124 (ESR1), Hs.26225 (GABRP), Hs.504115 (TRIM29), Hs.1594 (CENPA), Hs.184339 (MELK), Hs.592049 (PLK1), Hs.370834 (ATAD2), Hs.470477 (PTP4A2), Hs.473583 (YBX1) and Hs.83758 (CKS2) can be identified in a breast tissue sample obtained from a pre-menopausal mammal. In a particular embodiment, at least one of the genes selected from the group consisting of Hs.208124 (ESR1) and Hs.26225 (GABRP) is identified in a pre-menopausal mammal. Pre-menopausal is a time before menopause, or the permanent physiological, or natural, cessation of menstrual cycles.

In still another embodiment, methods of the invention identify genes selected from the group consisting of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1), and Hs.99962 (SLC43A3).

In a further embodiment, the methods of the invention identify genes selected from the group consisting of Hs.125867 (EVL), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.59212I (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1); Hs.444118 (MCM6), Hs.470477 (PTP4A2) and Hs.473583 (YBX1).

In still another embodiment, the methods of the invention identify genes selected from the group consisting of Hs.208124 (ESR1), Hs.26225 (GABRP), Hs.480819 (TBC1D9), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs. 654961 (FUT8). Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

In another embodiment, the methods of the invention identify genes selected from the group consisting of Hs.591314 (GMPS), Hs.444118 (MCM6), Hs.26010 (PFKP), Hs.469649 (BUB1), Hs.437638 (XBP1), Hs.523468 (SCUBE2), Hs.95612 (DSC2) and Hs.125867 (EVL), which may predict or may be associated with a grade (e.g., grade 1, 2, 3, or 4) of the breast cancer.

The American Joint Committee on Cancer (AJCC) staging of breast cancer is based on a scale of 0-4, with 0 having the best prognosis and 4 having the worst. There are multiple sub-classifications within each Stage classification (Robbins and Cotran, Pathological Basis of Disease, 7^{th} ed., Kumar, V., et al. (eds), Elsevier Saunders (2005)). Patients that present with ductal carcinoma in situ (DCIS) or lobular carcinoma in situ (LCIS) are considered stage 0. An invasive carcinoma of less than about 2 cm in the greatest dimension and no lymph node involvement is considered Stage I. An invasive carcinoma of less than about 5 cm in the greatest dimension and about 1 to about 3 positive lymph nodes is considered Stage II. Stage III refers to an invasive carcinoma of less than about 5 cm in the greatest dimension and four or more axillary lymph nodes involved or to an invasive carcinoma no greater than about 5 cm in the greatest dimension with nodal involvement or to an invasive carcinoma with at least about 10 axillary lymph nodes involved or invasive carcinoma with involvement of ipsilateral internal lymph nodes or invasive carcinoma with skin involvement, chest wall fixation or inflammatory carcinoma. Stage IV refers to a breast carcinoma with distant metastases (Robbins and Cotran Pathological Basis of Disease, 7^{th} Edition, eds. V. Kumar, et al., A. K. Abbas and N. Fausto, Elsevier Saunders (2005)).

Clinical staging of breast cancer is an estimate of the extent of the cancer based on the results of a physical exam, imaging tests (e.g., x-rays, CT scans) and often biopsies of affected areas. Blood tests can also be used in staging.

Pathological staging can be done on patients who have had surgery to remove or explore the extent of the cancer, which can be combined with clinical staging (e.g., physical exam, imaging tests). In some cases, the pathological stage may be different from the clinical stage. For example, surgery may reveal that the cancer has spread beyond that predicted from a clinical exam.

Restaging is sometimes used to determine the extent of the disease if a cancer recurs after treatment. This is done to help decide what the best treatment option would be at this time.

The TNM Staging System can be employed to stage breast cancers. Different systems had been employed to stage cancers and sometimes different systems were used to stage the same type of cancer.

The American Joint Committee on Cancer (AJCC) developed the TNM classification system as a tool for doctors to stage different types of cancer based on certain standard criteria. In the TNM system, each cancer is assigned a T, N, and M category (AJCC Cancer Staging Manual, 6^{th} ed., New York, Springer (2002)).

The T category describes the original, also referred to as “primary” tumor. The tumor size is usually measured in centimeters (about 2.5 centimeters or about 1 inch) or millimeters (about 10 millimeters or about 1 centimeter).
TX means the tumor can not be measured or evaluated.
T0 means there is no evidence of a primary tumor.
Tis means the cancer is in situ, or the tumor has not started growing into the structures around it.
The numbers T1-T4 describe the tumor size and/or level of invasion into nearby structures. The higher the T number, the larger the tumor and/or the further it has grown into nearby structures.

The N category describes whether or not the cancer has reached lymph nodes.
NX means the nearby lymph nodes can not be measured or evaluated.
N0 means nearby lymph nodes do not contain cancer.
The numbers N1-N3 describe the size, location, and/or the number of lymph nodes involved. The higher the N number, the more lymph nodes are involved.

The M category tells whether there are distant metastases or spread of cancer to other parts of the body.
MX means a metastasis can not be measured or evaluated.
M0 means that no distant metastases were found.
M1 means that distant metastases were found or the cancer has spread to distant organs or tissues.

Exemplary methods of stages of cancers include the following.

Once the T, N, and M are known, they are combined, and an overall “stage” of I, II, III, or IV is assigned. These stages may be subdivided, employing designations such as IIIA and IIIB). For example, a T1, N0, M0 breast cancer may indicate that the primary breast tumor is less than about 2 cm in the greatest diameter (T1), does not have lymph node involvement (N0) and has not spread to distant parts of the body (M0), which is a stage I cancer.

A T2, N1, M0 breast cancer would mean that the cancer is greater than about 2 cm but less than about 5 cm in its greatest diameter (T2), has reached only the lymph nodes in the underarm area (N1) and has not spread to distant parts of the body, which is a stage JIB cancer.

Stage I cancers are the least advanced and often have a better prognosis (also referred to as “outlook for survival”). Higher stage cancers (greater than stage I, for example, stage II, III or IV) are often more advanced and can, in many cases, be successfully treated. Stages of cancer take into account multiple components, including dimensions of the primary tumor, lymph node involvement and the presence of metastases.

Tumor grade is an assessment of the degree of differentiation in the cells within the tumor (Robbins and Cotran, Pathological Basis of Disease, 7^{th} ed., Kumar, V., et al. eds., Elsevier Saunders (2005)).

Tumor grade is considered when making treatment decisions and is another factor that affects prognosis for some kinds of cancer. The grade of the cancer reflects how abnormal the cancer cells look under the microscope. Grading is done by a pathologist who compares the cancer cells from the biopsy to normal cells. Grade is important because cancers with more abnormal-looking cells tend to grow and spread more quickly. Higher grade cancers (i.e., cancer cells look very abnormal) generally have a poor prognosis for survival and may require multiple and varied treatments.

The American Joint Committee on Cancer (ADCC) recommends the following cancer grading classifications:
GX: Grade cannot be determined
G1: Well-differentiated (the cancer cells look a lot like normal cells)
G3: Poorly differentiated (cancer cells don't look much like normal cells)
G4: Undifferentiated (the cancer cells don't look anything like normal cells)

The lower the tumor grade the better the prognosis. G1 cancers are linked to the best outcomes. G4 is associated with the worst outcomes and the others fall in between.

In an embodiment, the breast tissue sample is a grade 1 breast tissue sample in which methods of the invention identify at least one gene selected from the group consisting of Hs.591314 (GMPS), Hs.444118 (MCM6), Hs.26010 (PFKP), Hs.469649 (BUB1), Hs.437638 (XBP1), Hs.523468 (SCUBE2), Hs.95612 (DSC2) and Hs.125867 (EVL). In a particular embodiment, the methods of the invention identify in a stage 1 breast tissue sample at least one of genes is selected from the group consisting of Hs.26010 (PFKP), Hs.437638 (XBP1), Hs.444118 (MCM6) and Hs.469649 (BUB1).

In still another embodiment, the breast tissue sample is a grade 2 breast tissue sample in which methods of the invention identify at least one gene selected from the group consisting of Hs.591314 (GMPS), Hs.444118 (MCM6), Hs.26010 (PFKP), Hs.469649 (BUB1), Hs.437638 (XBP1), Hs.523468 (SCUBE2), Hs.95612 (DSC2) and Hs.125867 (EVL). In a particular embodiment, the methods of the invention identify in a stage 2 breast tissue sample as at least one of the gene Hs.125867 (EVL).

In yet another embodiment, the breast tissue sample is at least one member selected from the group consisting of a grade 3 breast tissue sample and a stage 4 breast tissue sample in which methods of the invention identify at least one gene selected from the group consisting of Hs.591314 (GMPS), Hs.444118 (MCM6), Hs.26010 (PFKP), Hs.469649 (BUB1), Hs.437638 (XBP1), Hs.523468 (SCUBE2), Hs.95612 (DSC2) and Hs.125867 (EVL). In a particular embodiment, at least one of the genes is selected from the group consisting of Hs.523468 (SCUBE2), Hs.95612 (DSC2) and Hs.591314 (GMPS) is identified in at least one member selected from the group consisting of a grade 3 breast tissue sample or a grade 4 breast tissue sample.

In an embodiment, one of the genes identified in the breast tissue sample is Hs.532824 (MAPRE2).

In another embodiment, one of the genes identified in the breast tissue sample is Hs.370834 (ATAD2). The breast tissue sample can include homogenates of tumor or breast biopsies, which include populations of different cell types (e.g., epithelial, stromal, smooth muscle).

In one embodiment, the breast tissue sample is a laser capture microdissection (LCM) breast tissue sample. LCM is known in the art and is described herein infra. LCM can result in collections of varying cell types (e.g., epithelial, stromal, smooth muscle) in varying numbers, such as 100 cells, 1000 cells, 2000 cells or 5000 cells. LCM can be employed to prepare a breast tissue sample that includes relatively pure populations of a single cell type, such as an epithelial cell, a stroma cell or a smooth muscle cell.

In another embodiment, the breast tissue sample is an intact tissue section breast tissue sample. Intact tissue section can be prepared employing established techniques. For example, an intact tissue section can be prepared by freezing a breast tissue sample obtained from a biopsy in O.C.T. (Optimum Cutting Temperature) and cryo-sectioning the intact breast tissue sample. The frozen intact tissue section is then placed on a glass slide and stained with hematoxylin and eosin to assess structural integrity. Additional frozen intact tissue sections are prepared for total RNA extraction, purification and analyzed by quantitative polymerase chain reaction (qPCR), as described infra.

Expression of the genes can be identified by detecting mRNA for the genes or the protein product of the gene (see, for example, U.S. Patent Application Nos. US 2005/0095607, US 2005/0100933 and US 2005/0208500, the teachings of all of which are hereby incorporated by reference in their entirety). The mRNA encoded by the genes and the gene product are indicated in Tables 1-36. Techniques to identify mRNA are known in the art and include, for example, qPCR, as described infra.

Expression of the genes in the methods described herein can be assessed by amplifying a nucleic acid sequence of the gene and detecting the amplified nucleic acid by well-established methods, such as the polymerase chain reaction (PCR), including quantitative PCR (qPCR), reverse transcription PCR (RT-PCR), and real-time PCR (including as a means of measuring the initial amounts of mRNA copies for each sequence in a sample), real-time RT-PCR or real-time Q-PCR. Exemplary techniques to employ such detection methods would include the use of one or two primers that are complementary to portions of a gene of interest (See Tables 1-36), where the primers are used to prime nucleic acid synthesis. The newly synthesized nucleic acids are optionally labeled and may be detected directly or by hybridization to a gene or mRNA. The newly synthesized nucleic acids may be contacted with polynucleotides of a breast tissue sample under conditions which allow for their hybridization. Additional methods to detect the expression of genes in the methods described herein include RNAse protection assays, including liquid phase hybridizations and in situ hybridization of cells.

The breast tissue sample can be from a primate mammal, such as a human. A patient is also a human mammal.

The methods described herein can further include the step of treating the mammal. For example, the methods of the invention may identify a mammal who has an increased likelihood of recurrence of an estrogen-receptor positive breast cancer, which may provide information for treating the mammal with, for example, compounds that block the action of the estrogen receptor, such as Tamoxifen, an orally active selective estrogen receptor modulator (Astra Zeneca Corporation). Similarly, the methods of the invention may identify a mammal who has an increased likelihood of recurrence of a grade 3 breast cancer, which may provide information about treating the mammal with, for example, medroxyprogesterone acetate or MEGACE®, synthetic progesterones that mimic the activity of progestin by binding progestin receptors.

Thus, the expression of the genes described herein may predict the survival and prognosis of the mammal. For example, the methods described herein identify a mammal who has an increased likelihood of recurrence of breast cancer, which may indicate an increased likelihood of death. Likewise, employing the methods described herein, a mammal may be identified who has a relatively low likelihood of recurrence of breast cancer, which may indicate increased survival.

The breast tissue sample can be a biopsy sample that includes at least one member selected from the group consisting of breast epithelial cells, breast stromal cells and breast smooth muscle cells. The breast tissue sample can be a breast biopsy that includes a carcinoma (ductal, lobular, medullary and/or tubular carcinoma) (also referred to as “carcinoma breast tissue sample”). The breast tissue sample can be a breast biopsy that includes stroma (also referred to as “stromal breast tissue sample”). The breast tissue sample can be subjected to laser capture microdissection (LCM) in which relatively pure populations of carcinoma cells (cancerous cells of breast epithelium) and/or relatively pure populations of stromal cells are obtained. “Relatively pure,” as used herein in reference to a carcinoma or stromal breast tissue sample, means that the sample is about 95%, about 98%, about 99% or about 100% one cell type (e.g., carcinoma or stroma).

The methods described herein may be used in combination with other methods of diagnosing breast cancer to thereby more accurately identify a mammal at an increased risk for recurrence of breast cancer. For example, the methods described herein may be employed in combination or in tandem with assessments of the presence or absence of estrogen and progestin steroid receptors, HER-2 expression/amplification (Mark H. F., et al. Genet Med 1:98-103 (1999)), Ki-67, an antigen that is present in all stages of the cell cycle except G0 and can be employed as a marker for tumor cell proliferation, and prognostic markers (including oncogenes, tumor suppressor genes, and angiogenesis markers) like p53, p27, Cathepsin D, pS2, multi-drug resistance (MDR) gene, and CD31. Alone or in combination with other clinical correlates of breast cancer, the methods described here may increase the accuracy of detection of breast cancer, in particular, in mammals who have had at least one or more incidents of breast cancer. In addition, such combinations of methods may increase the ability to accurately discriminate between various stages and/or grades of breast cancer. The methods described here may provide a means for predicting breast cancer survival outcomes and treatment regimens.

Increases (up-regulation of expression) and decreases (down-regulation of expression) of genes in the method described herein may be expressed in the form of a ratio between expression in a cancerous breast cell or a Universal Human Reference RNA (Stratagene, La Jolla, Calif.) (also referred to herein as a “control”) (See, for example, Table 36). For example, a gene can be considered up-regulated if the median expression value relative to a control, such as a Universal Human Reference RNA, is above one (1) (See, for example, Table 36). Likewise, a gene can be considered down-regulated if the median expression value relative to a control, such as a Universal Human Reference RNA, is less than one (1) (See, for example, Table 36).

Expression levels can be readily determined by quantitative methods as described herein. The methods described herein can identify over-expression (increases) or under-expression (decreases) of genes of Tables 1-36 compared to a Universal Human reference RNA control. Over-expression or under-expression can be correlated with patient characteristics (e.g., age, menopausal stage, disease-free) and breast cancer characteristics (e.g., grade stage, estrogen receptor status, progesterone receptor status).

Expression of the genes described herein can be assessed as a ratio of the expression of the gene in a breast tissue sample from the mammal and a control tissue sample, such as from another mammal with breast cancer, from a sample of the same mammal from a previous breast cancer incident, or a mammal without breast cancer (also referred to herein as “normal” or “non-cancerous”). For example, an increase in the ratio of expression of the gene in the breast tissue sample from the mammal compared to a non-cancerous sample, may indicate an increased likelihood of recurrence of the breast cancer. The ratios of increased expression can be about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 15, about 20, about 30, about 40, about 50, about 60, about 70, about 80, about 90, about 100, about 150, about 200, about 300, about 400, about 500, about 600, about 700, about 800, about 900 or about 1000. For example, a ratio of 2 is a 100% (or a two-fold) increase in expression. Likewise, a decrease in gene expression can be indicated by ratios of about 0.9, about 0.8, about 0.7, about 0.6, about 0.5, about 0.4, about 0.3, about 0.2, about 0.1, about 0.05, about 0.01, about 0.005, about 0.001, about 0.0005, about 0.0001, about 0.00005, about 0.00001, about 0.000005 or about 0.000001, which may indicate a decreased likelihood of recurrence of breast cancer in the mammal.

Similarly, increases and decreases in expression of the genes described herein can be expressed based upon percent or fold changes over expression in non-cancerous cells. Increases can be, for example, about 10, about 20, about 30, about 40, about 50, about 60, about 70, about 80, about 90, about 100, about 120, about 140, about 160, about 180 or about 200% relative to expression levels in non-cancerous cells. Alternatively, fold increases may be of about 1, about 1.5, about 2, about 2.5, about 3, about 3.5, about 4, about 4.5, about 5, about 5.5, about 6, about 6.5, about 7, about 7.5, about 8, about 8.5, about 9, about 9.5 or about 10 fold over expression levels in non-cancerous cells. Likewise, decreases may be of about 10, about 20, about 30, about 40, about 50, about 55, about 60, about 65, about 70, about 75, about 80, about 85, about 90, about 95, about 98, about 99 or 100% relative to expression levels in non-cancerous cells.

Exemplary methods to assess relative gene expression analyses include employing the ΔΔCt method, in which the threshold cycle number (C_{T} value) is the cycle of amplification at which the qPCR instrument system recognizes an increase in the signal (e.g., Sybr green florescence) associated with the exponential increase of the PCR product during the log-linear phase of nucleic acid amplification. These C_{T} values are compared to those of a housekeeping gene, such as glyceraldehyde phosphate dehydrogenase (GAPDH) or β-actin to obtain the ΔCt value, which is used to normalize for variation in the amount of RNA between different samples. The ΔCt value of each gene is then compared to that present in a calibrator, such as Universal Human Reference RNA (Stratagene, La Jolla, Calif.), in order to obtain a ΔΔCt value. Since each cycle of amplification doubles the amount of PCR product, the expression level of a target gene relative to that of the calibrator is calculated from 2^{-ΔΔCt}, expressed as relative gene expression.

In an additional embodiment, the invention is an immobilized collection (microarray) of the genes, such as a gene chip, described herein (Tables 1-36) for ease of processing in the methods described herein. The gene chips that include the genes described herein can permit high throughput screening of numerous breast tissue samples. The genes identified in the methods described herein can be chemically attached to locations on an immobilized collection, such as a coated quartz surface. Nucleic acids from breast tissue samples can be prepared as described herein and hybridized to the genes and expression of the genes identified.

The teachings of all patents, published applications and references cited herein are incorporated by reference in their entirety.

### EXEMPLIFICATION

### Example 1

A major health concern within the population of the United States today is breast cancer. This is due to the fact that it is the most prevalent form of cancer in women in the United States. The American Cancer Society estimates that 15 percent of cancer deaths in women will be due specifically to breast cancer, and it has the second highest mortality rate of all cancer types. It is estimated that 13.4 percent of women born in the United States today will be diagnosed with breast cancer at some point in their lives.

There has been tremendous progress toward understanding breast cancer, as well as other cancer types at both the molecular and genomic level, since the passing of the National Cancer Act in 1971. Certain tumor markers (e.g., estrogen and progestin receptors, HER-2/neu oncoprotein) in breast tissue biopsies have been used in clinical practice for evaluating a cancer patient's prognosis and therapy selection with success to a certain extent. The methods described herein are more accurate tests for diagnostics, prognostics, therapy selection, as well as monitoring response to treatment. Applications of genomic and proteomic approaches in studying human cancer can be complicated by the cellular heterogeneity of breast tissue biopsies.

Human tissue analyses present problems for developing clinically relevant and reliable genomic and proteomic testing. For example, analysis of the levels or activities of certain tumor markers to detect, diagnose or evaluate the prognosis of a cancer patient are currently performed either using biochemical or immunohistochemistry methodologies (Wittliff J L, et al., Steroid and Peptide Hormone Receptors Methods, Quality Control and Clinical Use, in Bland K I, Copeland III E M (eds); pp. 458-498, (1998); and Gelmann E P: Oncogenes in human breast cancer, in Bland K I, Copeland III E M (eds); pp. 499-517 (1998)). If the analyte is measured in a biochemical assay, a tissue biopsy consisting of a heterogeneous cell population is homogenized and the final concentration of the analyte from the cancer cells is reduced by the contamination of other proteins released from non-cancerous cells (e.g., normal stroma, epithelium and connective tissue cells). Therefore, a bias of the analyte concentration is likely to be observed due to the surrounding cell types, complicating the results obtained. Laser Capture Microdissection (LCM) can provide a rapid and straight-forward method for procuring homogeneous cells populations for biochemical and molecular biological analyses (Emmert-Buck M R, et al., Science 274:998-1001 (1996); Bonner et al. Science 278:1481-1483 (1997); and Simone N L, Trends in Genetics 14:272-276 (1998)).

Breast carcinoma tissue biopsies are not only composed of the carcinoma cells, but also of infiltrating endothelial cells, fibroblasts, macrophages, lymphocytes and other cells. The stroma surrounding the cancer cells provides the vascular support and extracellular matrix molecules that are required for tumor growth and progression (Shekhar M P, et al., Cancer Res 61:1320-1326 (2001)). Stromal cells may contribute to the developing tumor (Shekhar M P, et al., Cancer Res 61:1320-1326 (2001); Santner S J, et al. J Clin Endo Met 82:200-208 (1996); Matrisian L M, et al., Cancer Res 61:3844-3846 (2001); Mellick A S, et al., Int J Cancer 100:172-180 (2002); Fukino K, et al., Cancer Res 64:7231-7236 (2004); Schedin P, et al., Breast Cancer Res 6:93-101 (2004); and Tang Y, et al., Mol Cancer Res 2:73-80 (2004)). Differences in gene expression between breast carcinoma cells and the surrounding stromal cells may aid in the understanding of stromal responses to the presence of a tumor. The stroma may be an important target to control the malignant behavior of tumor cells that become resistant to standard therapies.

Studies have described “molecular signatures” of different cancer types, including breast cancer (Sgroi D C. et al., Cancer Res 59:5656-5661, (1999); Perou C M, et al., Nature 406:747-752 (2000); Wittliff J L, et al., Endocrine Soc Abs P3-198 (2002); van't Veer L J, et al., Nature 415:530-536 (2002); van de Vijver M J, et al., N Engl J Med 347:1999-2009 (2002); Kang Y, et al., Cancer Cell 3:537-549 (2003); Ma X J, et al., Breast Cancer Res Treat 82:S15 (2003); Ma X J, et al., Proc Natl Acad Sci USA 100:5974-5979 (2003); Ramaswamy S, et al., Nat Genet 33:49-54 (2003); Sortie T, et al., Proc Natl Acad Sci USA 100:8418-8423 (2003); Sotiriou C, et al., Proc Natl Acad Sci USA 100:10393-10398 (2003); Wittliff J L, et al., Jensen Symposium 2003 Abs. #64, p. 81 (2003); Ma X J, et al., Cancer Cell 5:607-616 (2004); Zhao H, et al., Mol Biol Cell 15:2523-2536 (2004); Jansen M P H M, J Clin Oncol 23:732-740 (2005); and Wang Y, et al., Lancet 365:671-679 (2005)). However, there has been great variation in the methods and microarray platforms utilized to obtain these profiles of cancer, including the use of breast cancer cell lines, intact tissue sections and LCM-procured cancer cells from tissue sections. The large gene sets implicated in cancer subtypes and progression identified in previous studies may have clinical relevance, but the number of genes to identify are too numerous for routine use in clinical management of patients. As described herein, data-mining has identified a smaller set of genes with equal or greater clinical application than predicted by those published studies that utilize hundreds or even thousands of genes. The gene subset was validated by qRT-PCR and evaluated for clinical utility in de-identified biopsies from breast cancer patients in the extensive IRS-approved Biorepository and Database (University of Louisville, Louisville, Ky.). The data described herein indicates that a) the gene expression profile of a gene subset exhibited by relatively pure carcinoma cell populations from a breast cancer biopsy more accurately predicts the recurrence status of a patient than currently used factors and b) the gene expression profile of surrounding normal stromal cells as opposed to those of carcinoma cells in a biopsy is related to the level of aggressiveness of the lesion, hence to the disease-free survival and overall-survival of the patient.

### Preparation and Handling of Human Tissue Biopsies

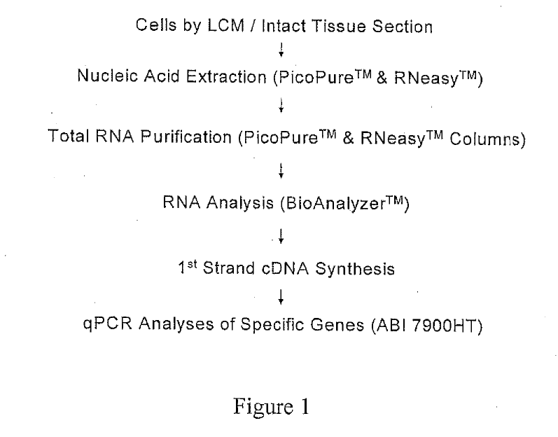

Previously established procedures for the preparation and handling of human tissue biopsies and subsequent isolation and processing of labile mRNA molecules from intact tissue sections and LCM-procured cells from frozen specimens for genomic analyses were employed (See, for example, Wittliff J L, et al., J Clin Ligand Assay 23:66 (2000) and Wittliff J L, et al., Methods Enzymol 356:12-25 (2002)). FIG. 1 is flow diagram that depicts the steps leading to validation and quantification of specific mRNA molecules, which are the expression products of genes. Briefly, mRNA was extracted from frozen breast tissue samples, intact tissue sections and from cells procured through laser capture microdissection (LCM).

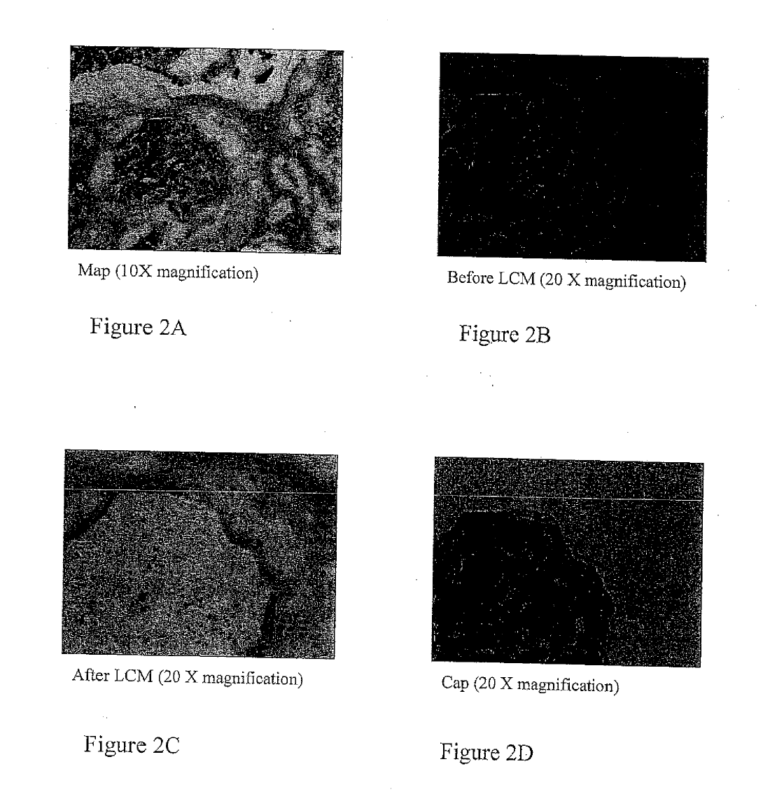

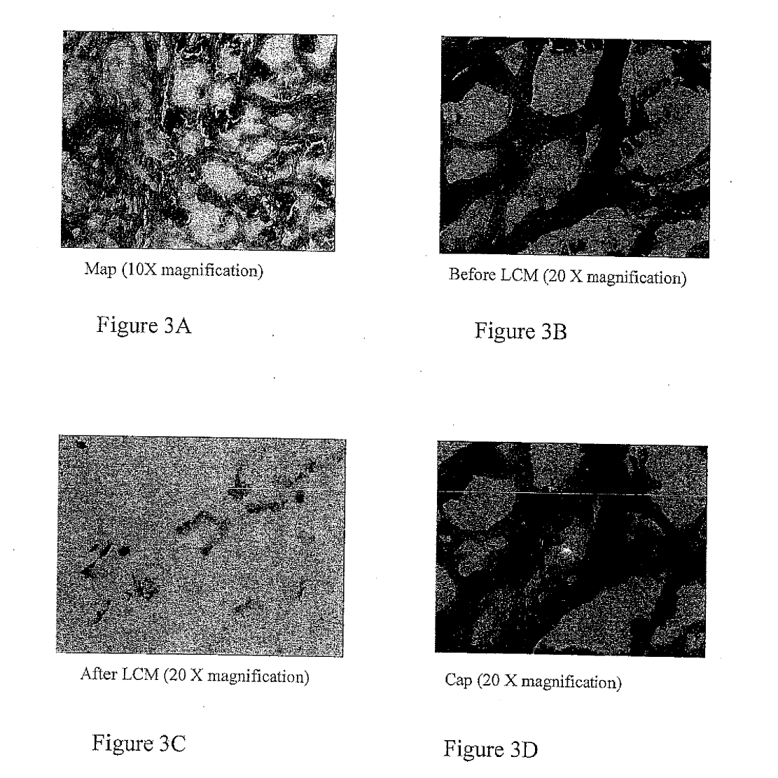

The PixCell IIe™ LCM System, sold by Arcturus Engineering, Inc., and the PixCell IIe™ Image Archiving Workstation were used to collect specific cell types, both normal and neoplastic under RNase-free conditions. Laser capture microdissection (LCM) is a major advancement in nondestructive cell sample technology. The cells of interest were microdissected using CapSure™ LCM Caps with the intact cells collected on the transfer film (FIGS. 2A-2D and 3A-3D). After cell collection DNA, RNA or proteins were extracted using a variety of established procedures.

Total RNA was isolated using commercially available kits, which were optimized for extracting RNA from de-identified cells procured by LCM. Intactness of RNA in de-identified intact tissue sections was evaluated prior to proceeding with LCM by a variety of procedures. For investigations of gene expression profiles of human tissues, cells of interest were procured (e.g., carcinoma or stromal) from different regions of a single de-identified tissue section. Carcinoma cells were removed from the regions of interest and procured on the LCM Caps (FIGS. 2D and 3D). Analyses were performed on whole tissue sections and LCM procured cells.

### Gene Expression

Expression of certain genes from breast carcinoma cells collected by LCM have been described (Ma X J, et al., Breast Cancer Res Treat 82:S15 (2003); Wittliff J L, et al., Jensen Symposium, Abs. #64, p. 81 (2003); U.S. Pub. No. 2005/0208500; U.S. Pub. No. 2005/0095607; U.S. Pub. No. 2005/0100933; Emmert-Buck M R, et al., Science 274:998-1001 (1996); Bonner R F, et al., Science 278:1481-1483 (1997); Simone N L, et al., Trends in Genetics 14:272-276 (1998); Shekhar M P, et al., Cancer Res 61:1320-1326 (2001); Santner S J, et al., J Clin Endo Met 82:200-208 (1996); Matrisian L M, et al., Cancer Res 61:3844-3846 (2001); Mellick A S, et al., Int J Cancer 100:172-180 (2002); Fukino K, et al., Cancer Res 64:7231-7236 (2004); Schedin P, et al., Breast Cancer Res 6:93-101 (2004); Tang Y, et al., Mol Cancer Res 2:73-80 (2004); and Sgroi D C, et al., Cancer Res 59:5656-5661 (1999)).

GenBank Accession numbers (NCBI) (van't Veer L J, et al., Nature 415:530-536 (2002); van de Vijver M J, et al., N Engl J Med 347:1999-2009 (2002); Kang Y, et al., Cancer Cell 3:537-549 (2003); Ma X J, et al., Breast Cancer Res Treat 82:S15 (2003); Ma X J, et al., Proc Natl Acad Sci USA 100:5974-5979 (2003); Ramaswamy S, et al., Nat Genet 33:49-54 (2003); Sorlie T, et al., Proc Natl Acad Sci USA 100:8418-8423 (2003); Sotiriou C, et al., Proc Natl Acad Sci USA 100:10393-10398 (2003); Wittliff J L, et al., Jensen Symposium, Abs. #64, p. 81 (2003); Ma X J, et al., Cancer Cell 5:607-616 (2004); Jansen M P H M, et al., J Clin Oncol 23:732-740 (2005); and Wang Y, et al., Lancet 365:671-679 (2005)) were entered into the UniGene database (NCBI), which separates the GenBank sequences into a non-redundant set of gene-oriented clusters. Currently, there are about 122,987 sequence entries for Homo sapiens. Each UniGene Cluster contains sequences that represent a unique gene, which has a specific identifier. Once the appropriate UniGene identifier is known, the gene sets can be sorted by the UniGene identifier and analyzed. For example, epidermal growth factor receptor (EGFR) has a GenBank Accession number of NM_{—}201284. Entry of this Accession number into the UniGene database identifies UniGene Cluster Hs.488293 Homo sapiens Epidermal growth factor receptor (erythroblastic leukemia viral (v-erb-b) oncogene homolog, avian) (EGFR). Twenty-four mRNA sequences have been entered including NM_{—}201284 for EGFR. In addition 335 expressed sequence tag (EST) sequences have been entered.

Once the UniGene identifiers were compiled into a Microsoft Excel spreadsheet, they were imported into Microsoft Access and analyzed collectively. A Tier 1 level of comparison identified any gene that appeared in at least 2 molecular signatures, while a Tier 2 comparison identified any gene that appeared in at least 3 signatures. T0 identify genes that appear most relevant in breast carcinoma cells compared to those of surrounding stromal cells, the Tier 2 genes were separated into two groups. The genes were analyzed employing relatively pure (e.g., about 95%, about 98%, about 99% or 100%) carcinoma cells and/or relatively pure (e.g., about 95%, about 98%, about 99% or 100%) stromal cells.

Eleven (11) molecular signatures of about 2604 genes were analyzed (van't Veer L J, et al., Nature 415; 530-536 (2002); Kang Y, et al., Cancer Cell 3:537-549 (2003); Ma X J, et al., Breast Cancer Res Treat 82:S15 (2003); Ma X J, et al., Proc Natl Acad Sci USA 100:5974-5979 (2003); Ramaswamy S, et al., Nat Genet 33:49-54, (2003); Sadie T, et al., Proc Natl Acad Sci USA 100:8418-8423 (2003); Sotiriou C, et al., Proc Natl Acad Sci USA 100:10393-10398 (2003); Wittliff J L, et al., Jensen Symposium, Abs. #64, p. 81 (2003); Ma X J, et al., Cancer Cell, 5:607-616 (2004); Jansen M P H M, et al., J Clin Oncol, 23:732-740 (2005); Wang Y, et al., Lancet, 365:671-679 (2005)). About 354 of these genes were identified in at least two of the signatures and 32 genes subsequently identified. Fourteen (14) of the genes identified were relatively pure carcinoma cells obtained by LCM (Table 1). The remaining 18 genes were relatively pure carcinoma cells (Table 1). Surrounding cells may be important in cancer progression. These 32 genes may include genes that contribute to the growth behavior of the cancer.

TABLE 1
UniGene Identifier, Gene Description and mRNA Accession
Number

| UniGene | | mRNA Accession |
|---|---|---|
| Identifier | Gene Description | Number |
| Hs.125867* | EVL | NM_016337.2 |
| | Enah//Vasp-like | |
| Hs.591847* | NAT1 | NM_000662.4 |
| | N-acetyltransferase 1 | |
| | (arylamine n-acetyltransferase) | |
| Hs.208124* | ESR1 | NM_000125.2 |
| | Estrogen Receptor 1 | |
| Hs.26225* | GABRP | NM_014211.1 |
| | Gamma-aminobutyric acid | |
| | (GABA) A receptor, pi | |
| Hs.408614* | ST8SIA1 (SIAT8A) | NM_003034.3 |
| | ST8 alpha-N-acetyl- | |
| | neuraminide alpha-2,8- | |
| | sialytransferase 1 | |
| Hs.480819* | TBC1D9 (KIAA0882) | NM_015130.2 |
| | TBC1 domain family, member | |
| | 9 (with GRAM domain) | |
| Hs.504115* | TRIM29 | NM_012101.3 |
| | Tripartitie motif-containing 29 | |
| Hs.523468* | SCUBE2 | NM_020974.1 |
| | Signal peptide, CUB domain, | |
| | EGF-like 2 | |
| Hs.532082* | IL6ST | NM_002184.2 |
| | Interleukin 6 signal transducer | |
| | (gp130, oncostatin M receptor) | |
| Hs.592121* | RABEP1 | NM_004703.4 |
| | Rabaptin, RAB GPTase | |
| | binding effector protein 1 | |
| Hs.79136* | SLC39A6 | NM_012319.3 |
| | Solute carrier family 39 (zinc | |
| | transproter), member 6 | |
| Hs.82128* | TPBG | NM_006670.3 |
| | Trophoblast glycoprotein | |
| Hs.95243* | TCEAL1 | NM_004780.2 |
| | Transcription elongation factor | |
| | A (SII)-like1 | |
| Hs.95612* | DSC2 | NM_024422.2 |
| | Desmocollin 2 | |
| Hs.654961 | FUT8 | NM_004480.3 |
| | Fucosyltransferase 8 (alpha | |
| | (1,6) fucosyltransferase) | |
| Hs.1594 | CENPA | NM_001809.3 |
| | Centromere protein A | |
| Hs.184339 | MELK | NM_014791.2 |
| | Maternal embryonic leucine | |
| | zipper kinase | |
| Hs.26010 | PFKP | NM_002627.3 |
| | Phosphofructokinase, platelet | |
| Hs.592049 | PLK1 | NM_005030.3 |
| | Polo-like kinase 1 | |
| Hs.370834 | ATAD2 | NM_014109.3 |
| | ATPase family, AAA domain | |
| | containing 2 | |
| Hs.437638 | XBP1 | NM_005080.2 |
| | X-box binding protein 1 | |
| Hs.444118 | MCM6 | NM_005915.4 |
| | MCM6 minichromosome | |
| | maintenance deficient 6 | |
| Hs.469649 | BUB1 | NM_004336.2 |
| | BUB1 budding uninhibited by | |
| | benzimidazoles 1 homolog | |
| Hs.470477 | PTP4A2 | NM_080392.2 |
| | Protein tyrosine phosphatase | |
| | type IVA, member 2 | |
| Hs.473583 | YBX1 | NM_004559.3 |
| | Y box binding protein 1 | |
| Hs.480938 | LRBA | NM_006726.2 |
| | LPS-responsive vesicle | |
| | trafficking, beach and anchor | |
| | containing | |
| Hs.524134 | GATA3 | NM_002051.2 |
| | GATA binding protein 3 | |
| Hs.531668 | CX3CL1 | NM_002996.3 |
| | Chemokine (C—X3—C motif) | |
| | ligand 1 | |
| Hs.532824 | MAPRE2 | NM_014268.1 |
| | Microtubule-associated protein, | |
| | RP/EB family, member 2 | |
| Hs.591314 | GMPS | NM_003875.2 |
| | Guanine monphosphate | |
| | synthetase | |
| Hs.83758 | CKS2 | NM_001827.1 |
| | CDC28 protein kinase | |
| | regulatory subunit 2 | |
| Hs.99962 | SLC43A3 | NM_199329.1 |
| | Solute carrier family 43, | |
| | member 3 | |

*indicates genes from studies utilizing LCM-procured carcinoma cells

### Quantitative Polymerase Chain Reaction

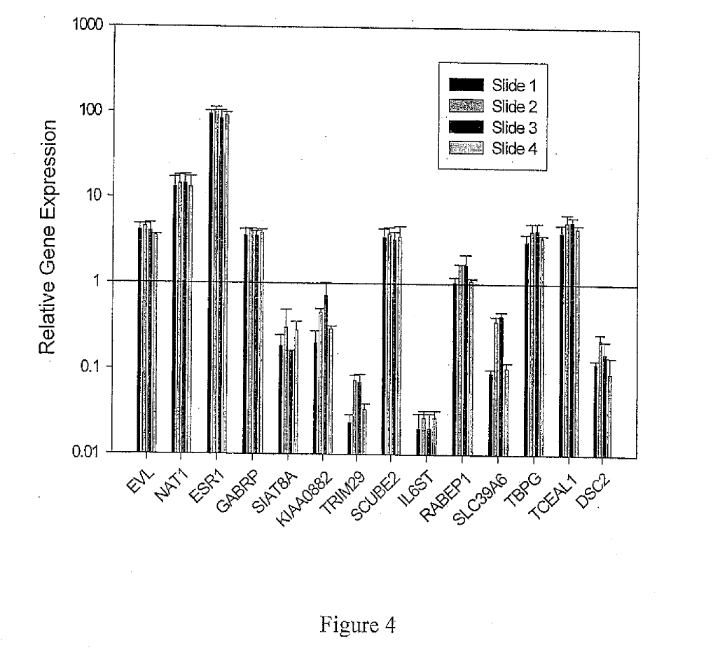

Real-time quantitative polymerase chain reaction (qPCR) using the ABI Prism 7900HT system (Applied Biosystems) was utilized to analyze and validate the expression of these 32 genes of Table 1. This method allows quantitative examination of the gene transcripts of interest (FIG. 4). Cells from the preparations of gross de-identified tissue sections and LCM-procured cells were lysed and the extracts examined for target gene transcription. RNA from each cell type was extracted and reverse transcribed to cDNA prior to qPCR analyses.

In order to relate the results from qPCR measurements of the level of expression of the gene subset with tumor marker analyses, patient characteristics (e.g., age, menopausal status), tumor properties (e.g., pathology, grade) and clinical outcome (e.g., disease-free and overall survival) were analyzed using several statistical analyses (e.g., T-tests, Anova, Kaplan-Meir, Cox Regression). Using the IRB-approved Biorepository and Database of the Hormone Receptor Laboratory, de-identified samples of primary invasive ductal carcinoma were examined. Tissue-based properties (e.g., pathology of the cancer, grade, and size) and encoded patient-related characteristics (e.g., age, race, menopausal status, nodal status, clinical treatment and response) were utilized to examine the relationship between gene expression results and clinical parameters.

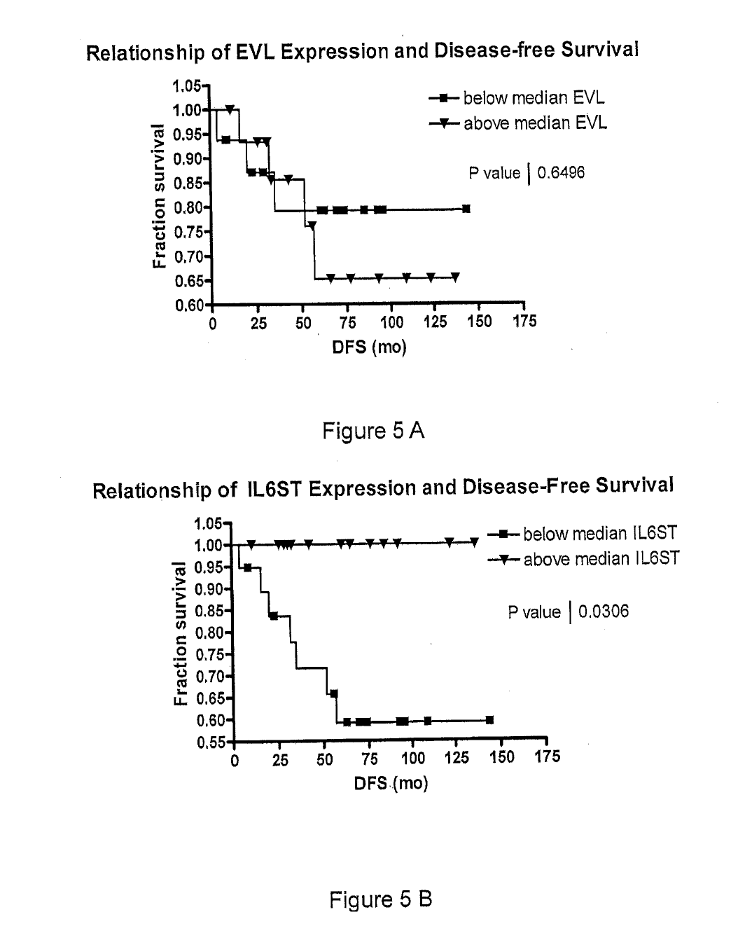

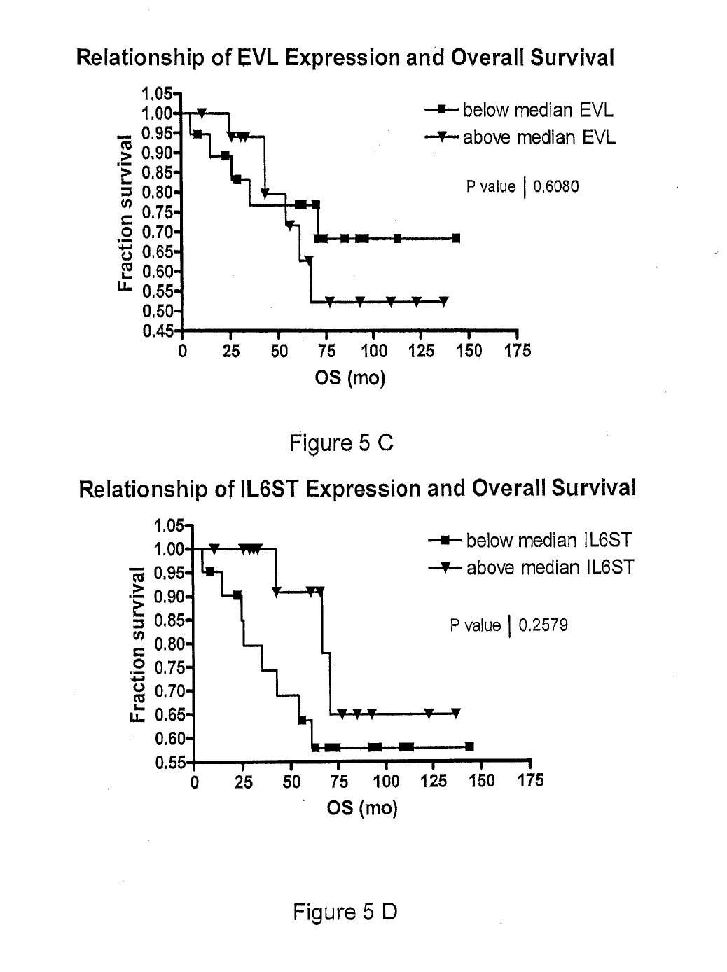

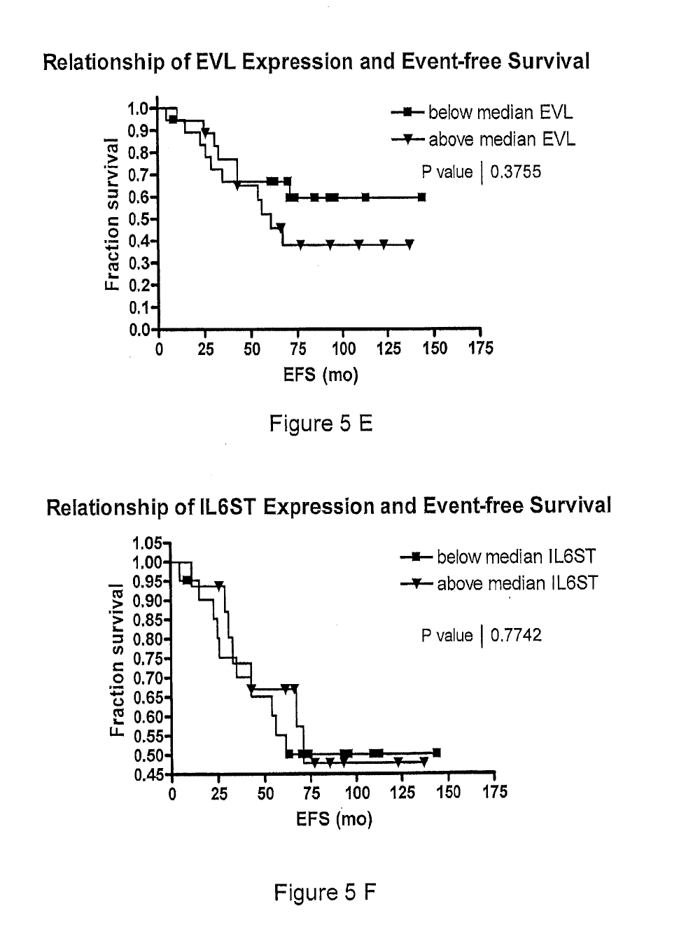

The gene expression data were correlated with de-identified patient characteristics and clinical data that are present in the Hormone Receptor Laboratory Tumor Marker™ Database. Gene expression was analyzed by Kaplan-Meier survival plots using GraphPad Prism™ software. This software allows a statistical analysis of gene expression and its association with recurrence of the cancer (disease-free survival—DFS), death of the patient due to that cancer (overall survival—OS), and death by any means (event-free survival—EFS) (FIG. 5A-5F). Expression of each gene was then evaluated for expression above and below median relative expression values (FIGS. 5A-5F). The expression of many genes depicted in, for example, Tables 4 and 7 showed correlations with recurrence and survival when tested individually, while others appeared to indicate trends which separated patients into groups. Of the 14 genes evaluated in a carcinoma gene subset, 8 genes (CENPA, DSC2, GABRP, GATA3, MAPRE 2, RABEP1, SCUBE2, SLC43A3) appear to be associated with either recurrence or survival with correlation coefficients less than 0.20 when evaluated individually. Three of the genes in the subset independently appear to predict recurrence or survival with a correlation coefficient less than 0.05. These studies were performed by analyzing the expression of each gene individually; and correlating it with clinical outcome. However, there is more likely greater power of prediction when the genes are analyzed collectively.

Not all of the genes tested showed correlations with recurrence and survival, but some appear to indicate trends which separate patients into groups. Of the 32 genes evaluated in the gene subsets, 8 genes appear to be moderately associated with either recurrence or overall survival with a P value less than 0.20. Only one of the genes (SLC43A3) individually predicted recurrence or overall survival with a P value less than 0.05. The Hazard Ratios for each gene are shown (Table 5), but it should be noted that these are only representative of the gene once defined significant. These analyses could also be completed using expression data of the subset genes from the previous microarray study. Since 247 patients were evaluated in that study, there may be greater statistical significance within the larger sample population. Similar evaluations using the LCM-procured pure cell populations will also be performed, although with a smaller sample size.

### Example 2

The large gene sets utilized to determine cancer subtypes and outcome prediction identified in previous studies are much too numerous for routine use in clinical management of patients. By data-mining the studies described in Example 1, a smaller gene set has been compiled with greater clinical utility than predicted by those studies that utilize hundreds or even thousands of genes. This gene set can be validated, tested and analyzed for clinical utility in breast cancer patients. It is believed that the expression profile of a gene subset exhibited by either an intact tissue section or a preparation of relatively pure carcinoma or relatively pure stromal cells from a breast cancer biopsy more accurately predicts the clinical course (e.g., disease-free survival and overall-survival) of a patient than predicted by currently used factors (e.g., ER/PR status, stage, grade, nodal status and size of the tumor).

qPCR analyses were used to evaluate expression of mRNA isolated from intact tissue sections to identify expression of the gene subsets derived above. The qPCR results can used to compare gene expression levels in a selected number of paired samples (e.g., intact and LCM-procured cells from serial tissue sections) to ascertain the contribution of cellular heterogeneity.

As described above in Example 1, real-time qPCR using the ABI Prism 7900HT system (Applied Biosystems) was utilized. This method allows quantitative examination of the gene transcripts of interest. Cells from the preparations of gross tissue sections and LCM-procured cells were lysed, and the extracts were examined for target gene transcription. RNA from each cell type was extracted and isolated with the Arcturus PicoPure™ (for LCM-procured cells) or Qiagen RNeasy™ RNA isolation kit (for intact tissue section analyses). Total RNA was then reverse transcribed to cDNA prior to qPCR.

Before analyses of gene expression in tissue specimens, extensive quality control experiments were performed.

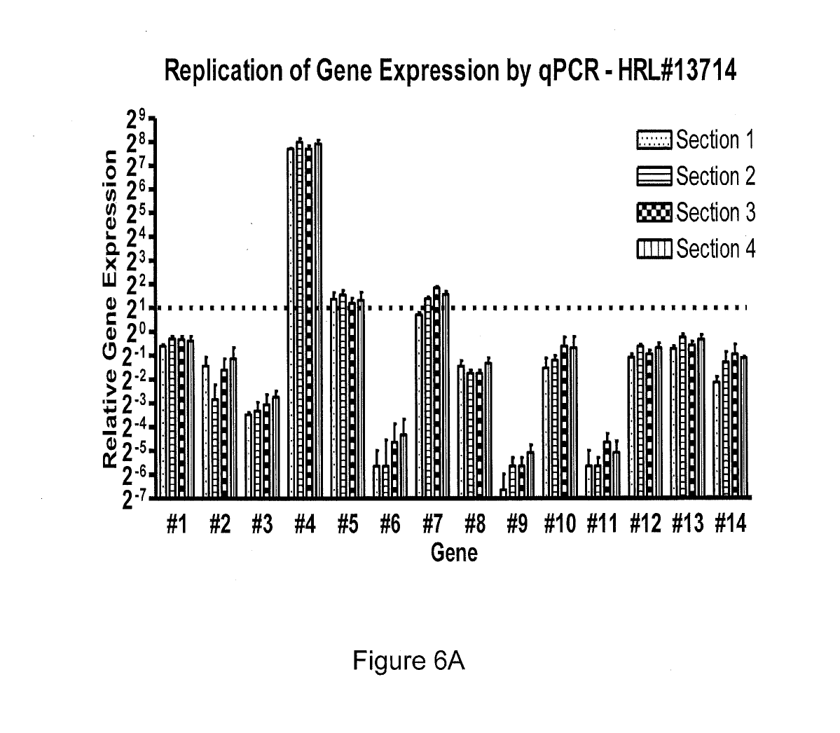

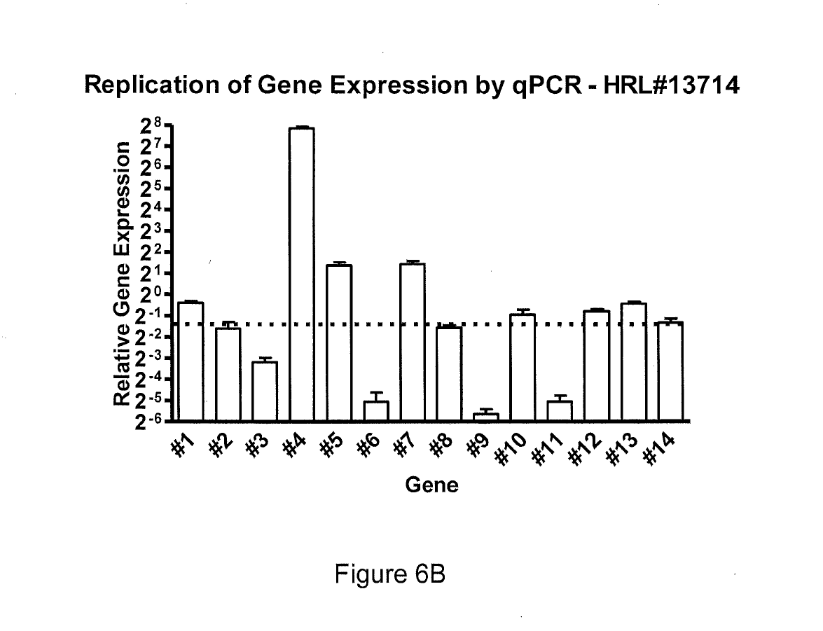

In one quality control experiment, preparation of 4 sections from each of 3 specimens were analyzed. These sections were processed concurrently, through scraping, RNA isolation, reverse transcription and qPCR of the 14 genes (Table 1, Table 15) in the carcinoma subset. The qPCR reactions were performed in triplicate with duplicate wells in each 384-well plate, with the level of reproducibility illustrated (FIGS. 6A and 6B). As shown in FIG. 6B, the collective results from 12 analyses are highly reproducible supporting this validation approach.

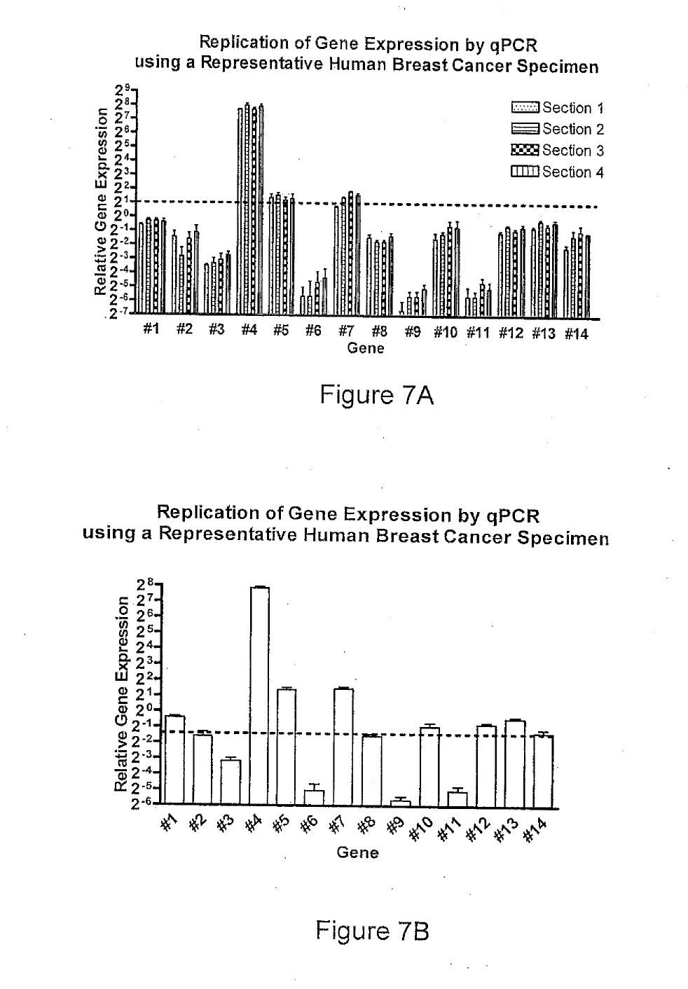

In another quality control test three tissue sections were analyzed. Each tissue section was processed and evaluated independently on different days to ascertain inter-assay variation. Each specimen was analyzed by qPCR in triplicate with duplicate wells in each 384-well plate. The data were then evaluated and compared between tissue sections (FIG. 7A) as well as between each qPCR run (FIG. 7B). These data also provided evidence that measurements of gene expression levels of each specimen were reproducible

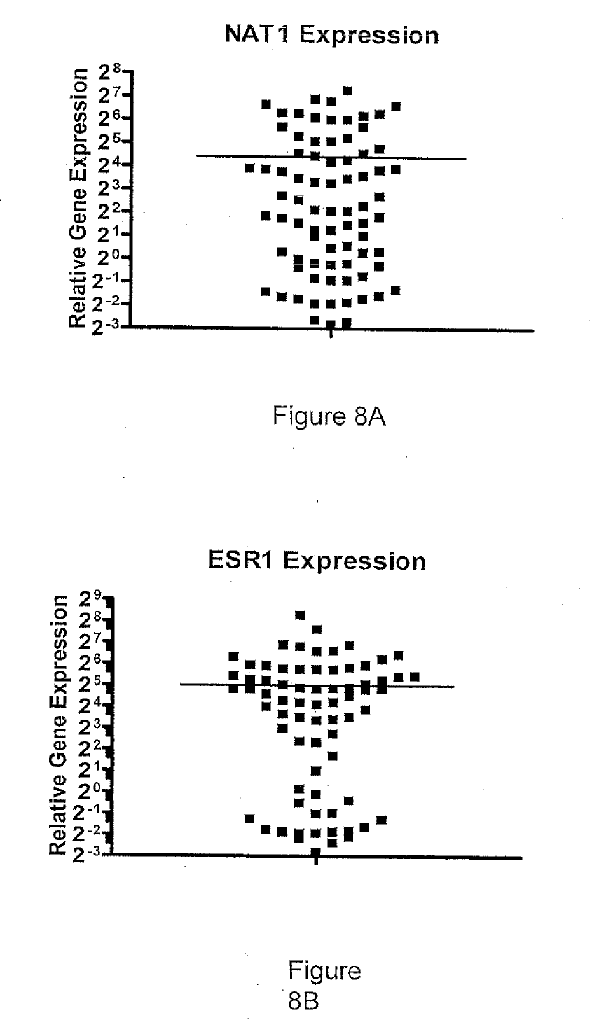

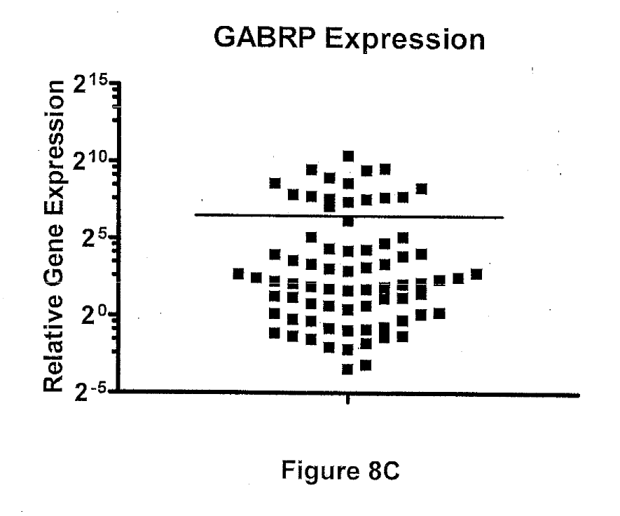

After achieving reproducible results with the quality control experiments, 78 intact tissue section were analyzed in triplicate experiments for the expression of the 32 genes (Table 1) in both the carcinoma cell and stromal cell subsets. These results were plotted to visualize the distribution and range of expression levels of each gene (FIGS. 8A-8C). If there appeared to be a bimodal distribution, the difference in those groups were investigated as a potential biomarker. Two (2) of the 32 genes (Hs.208124 (ESR1) and Hs.26225 (GABRP)) examined in both gene subsets have a modest grouping of expression levels. These specimens can be analyzed using both gene subsets in order to obtain statistical significance related to patient characteristics as described below.

The gene subsets (Table 1, Table 15) derived earlier also are being analyzed using LCM-procured relatively pure cell populations. Many specimens having carcinoma and stromal cells isolated by LCM are available for analysis. Of the samples isolated by LCM, 15 have been analyzed for each cell type with qPCR of the corresponding gene sets. After isolation, the RNA is was first evaluated with the BioAnalyzer™ (Agilent Technologies) for quality and semi-quantification before proceeding to reverse transcription and qPCR. Multiple LCM caps (about 2 to about 3 LCM caps) were pooled to obtain a greater quantity of RNA, so that a linear amplification step is not necessary prior to qPCR. The target amount of RNA from LCM-procured cells for a qPCR reaction is 10 ng from carcinoma cells and 1 ng from stromal cells. For control purposes, the concentration of Universal Human Reference RNA (Stratagene) is adjusted to be similar to that of the experimental reactions in the plate.

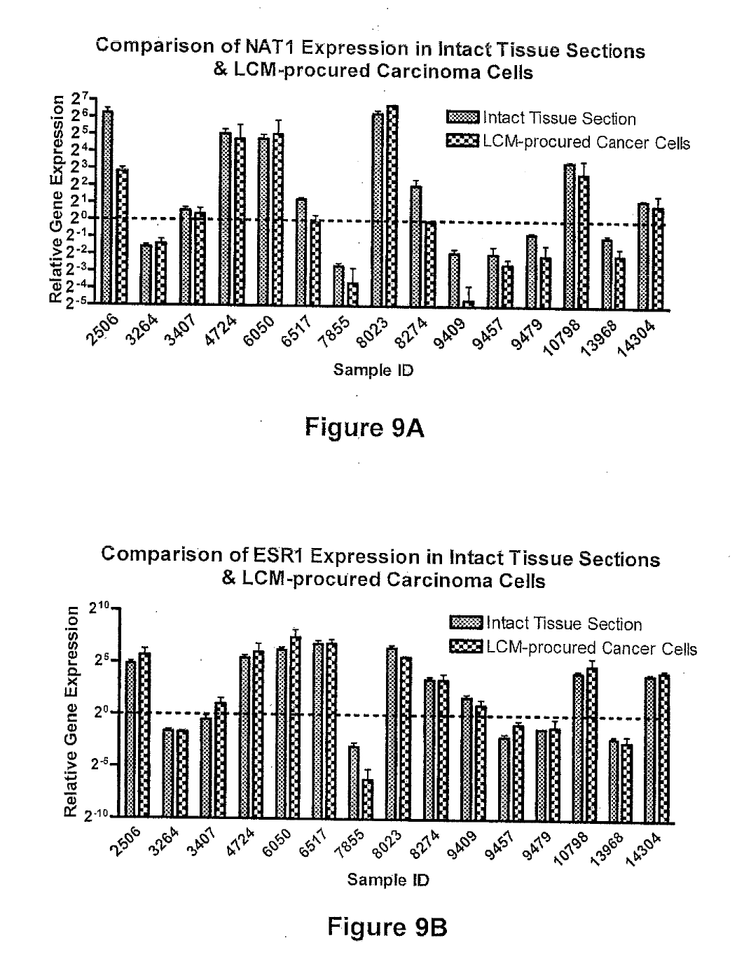

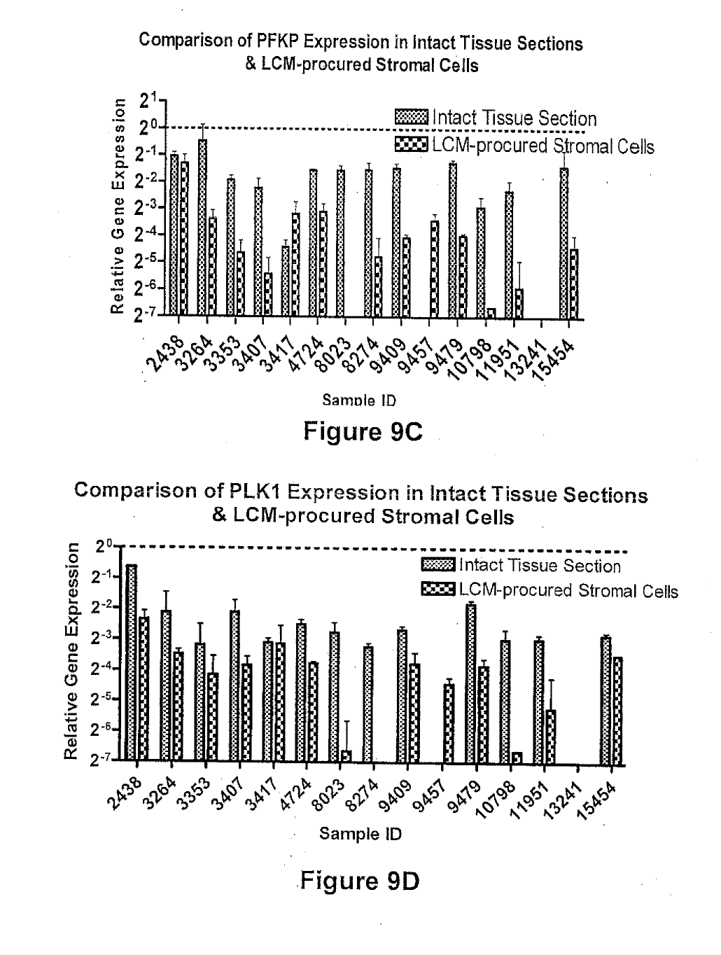

Gene expression was compared between the intact tissue section and LCM-procured cell populations corresponding to the two gene subsets (FIGS. 9A-9D) and paired t-tests were used to identify any gene in which the expression was significantly different between the cells procured from intact tissue sections versus LCM (Table 2).

TABLE 2
Results of paired t-tests illustrating differences in gene expression
between intact tissue sections and LCM-procured cells.

| Gene ID | P-Value | Gene ID | P-Value |
|---|---|---|---|
| EVL | 0.0924 | FUT8 | 0.1386 |
| NAT1* | 0.5528 | CENPA | 0.0024 |
| ESR1* | 0.2971 | MELK | 0.0141 |
| GABRP | 0.0577 | PFKP* | 0.0001 |
| ST8SIA1 | 0.0887 | PLK1* | 0.0009 |
| TBC1D9 | 0.0664 | ATAD2 | 0.0032 |
| TRIM29 | 0.4743 | XBP1 | 0.0108 |
| SCUBE2 | 0.0710 | MCM6 | 0.0179 |
| IL6ST | 0.1964 | BUB1 | 0.0070 |
| RABEP1 | 0.1140 | PTP4A2 | 0.0309 |
| SLC39A6 | 0.0814 | YBX1 | 0.0045 |
| TPBG | 0.5763 | LRBA | 0.4280 |
| TCEAL1 | 0.1448 | GATA3 | 0.1837 |
| DSC2 | 0.6705 | CX3CL1 | 0.0241 |
| | | MAPRE2 | 0.4824 |
| | | GMPS | 0.0297 |
| | | CKS2 | 0.1232 |
| | | SLC43A3 | 0.0031 |

*indicates data shown in FIGS. 9A-9D.

Gene expression from the carcinoma cells subset corresponded well between the intact tissue section and LCM-procured cancer cells (none statistically different), further supporting the selection approach of the candidate gene subset.

However, genes in the relatively pure stromal cell subset appeared to exhibit much greater differences in expression between the two groups (13 genes with P values <0.05). In general, gene expression was statistically different in that gene expression levels were lower in LCM-procured stromal cells compared to intact tissue sections. This may be an artifact due to the small concentration of stromal cell RNA analyzed (e.g., average amount of RNA analyzed was about 2.6 ng), where Ct values were in the low to mid 30 s. This can be addressed by increasing the amount of RNA obtained for analysis.

One conclusion that could be drawn to explain these differences in gene expression in the different cell types is that most of the samples analyzed are primarily composed of carcinoma cells, consequently there are likely few differences between the intact tissue sections and relatively pure carcinoma cells collected by LCM and because carcinoma cells produce much more RNA than the cells of the surrounding stroma, the stromal cell gene expression is masked in intact tissue analysis. Thus, LCM may be beneficial when studying gene expression in stromal cells, but not necessarily in carcinoma cells. The cellular composition of each individual tissue section should be taken into consideration.

Another set of experiments using LCM-procured cells populations to analyze the expression of the converse gene subset is made in order to determine if the two subsets indeed represent the two cell types. For example, if the “stromal gene subset” is really only clinically significant in the surrounding stromal cells, and not just statistically eliminated from prior analysis of the molecular signatures.

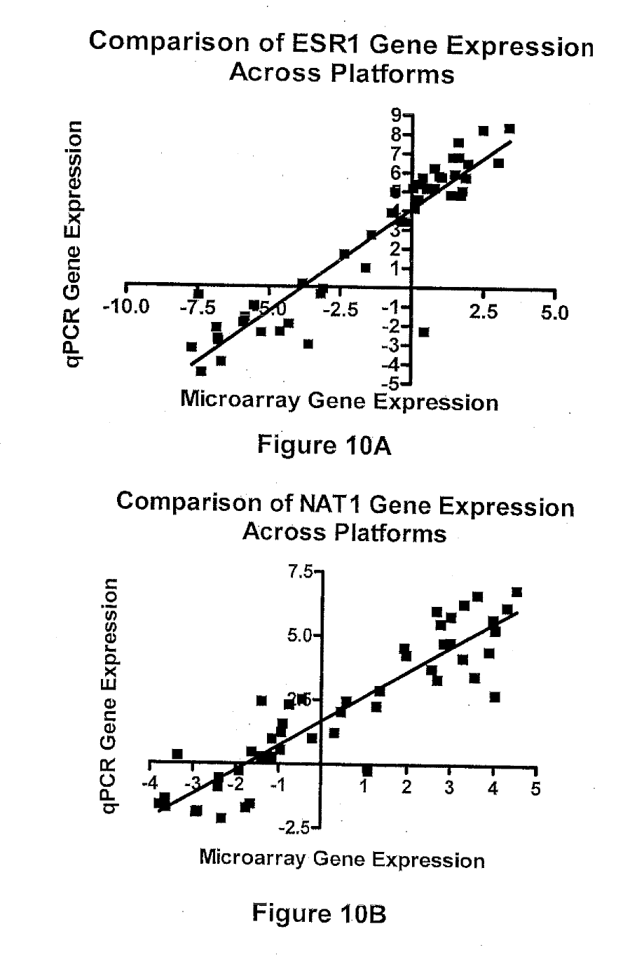

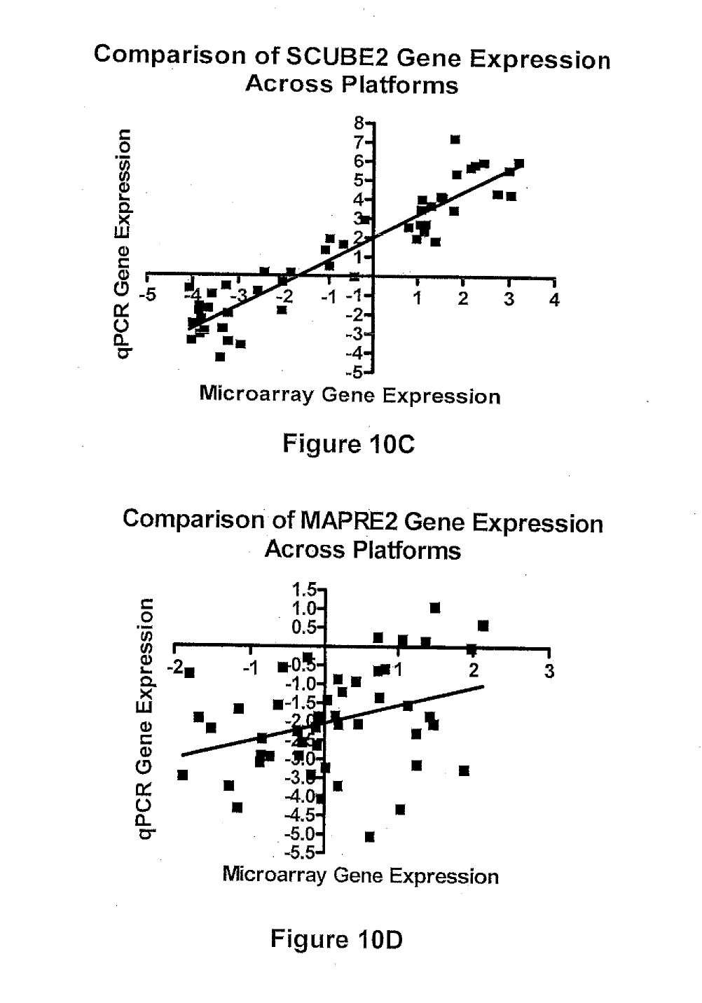

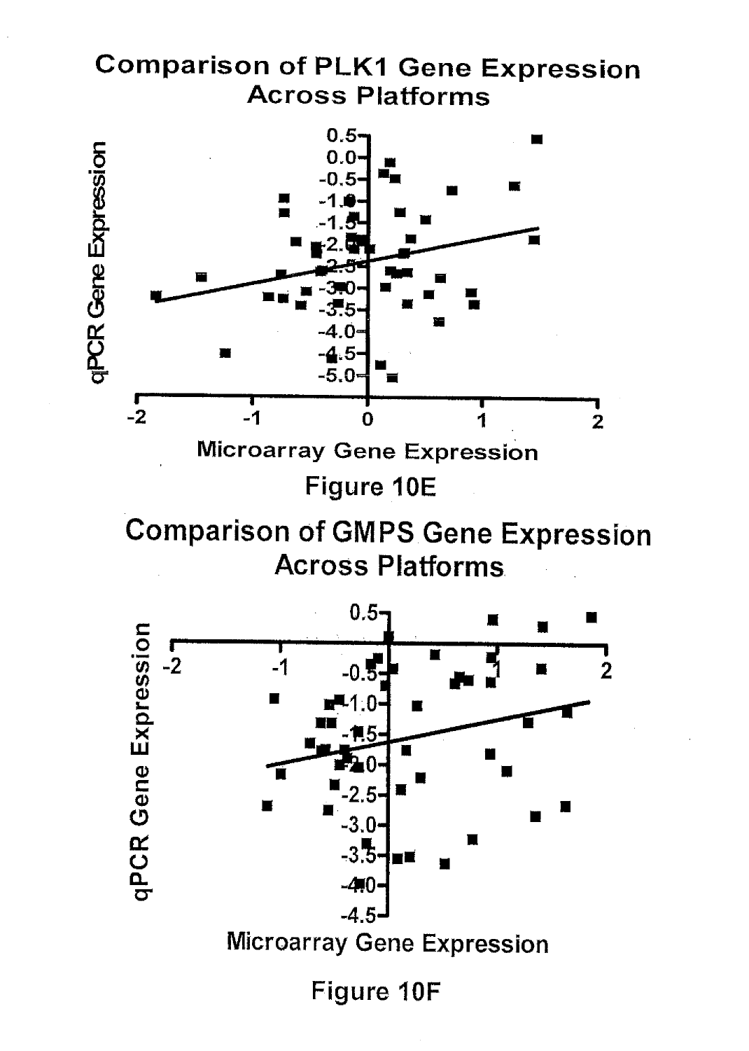

An analysis of 48 specimens has been performed comparing the qPCR gene expression from intact tissue to the microarray data obtained from LCM-procured carcinoma cells (FIGS. 10A-10F, Table 3). These 48 specimens were obtained from a total of 78 specimens. This will not only allow comparisons of gene expression data across platforms (comparing microarray data and qPCR data), but will also provide insight as to whether LCM is necessary for gene expression studies focusing on clinical relevance, i.e., if whole tissue-derived data are providing the same information as obtained from LCM, then the additional steps and reagents are unnecessary. This analysis may be complicated by different cell types present in a sample, and additional data incorporating histology data may be also need to be analyzed, i.e., percent carcinoma, stromal and inflammatory cells.

These comparisons are also interesting because of correlations among genes from the stromal cell subset. Certain genes within the stromal cell subset may be expressed in both cell types or only in carcinoma cells (e.g., Hs.437638 (XBP1) and Hs.524134 (GATA3) correlated to respective microarray data with an r² value of 0.7). These genes may have been filtered from molecular signatures based on the statistical algorithm used.

Generally, genes from carcinoma cells subset correlate better with the microarray data than the genes from the stromal cell subset, and a t-test between correlation coefficients (r² values) from the genes within the two subsets provides a p-value of 0.0013, indicating that there is a difference between the two groups. The three genes which correlated best with the microarray data are shown in the top row of Table 4 (i.e., genes from the cancer cell subset), while the three genes which correlated poorly with the microarray data are shown in the bottom row (i.e., genes from the stromal cell subset). The fact that some of the genes do not correlate well is not necessarily indicative of the influence of stromal cells, but could also be due to differences in platforms used, which is why this should be also tested directly by qPCR.

TABLE 3
Results from linear regression analyses of comparisons between gene
expression data obtained by qPCR and microarray.

| | | Slope of | P-Value (Is the | |
|---|---|---|---|---|
| | Gene | linear | slope significantly | |
| Gene ID | Subset | regression | non-zero?) | r² |
| ATAD2 | Stroma | 0.5 | <0.0001 | 0.29 |
| BUB1 | Stoma | 0.5 | 0.0027 | 0.18 |
| CENPA | Stroma | 0.72 | <0.0001 | 0.57 |
| CKS2 | Stoma | 0.67 | 0.0032 | 0.17 |
| CX3CL1 | Stroma | 0.51 | <0.0001 | 0.49 |
| DSC2 | Cancer | 0.79 | 0.0001 | 0.27 |
| ESR1* | Cancer | 1.1 | <0.0001 | 0.85 |
| EVL | Cancer | 1 | <0.0001 | 0.62 |
| FUT8 | Stoma | 0.96 | <0.0001 | 0.48 |
| GABRP | Cancer | 0.93 | <0.0001 | 0.60 |
| GATA3 | Stoma | 1.3 | <0.0001 | 0.70 |
| GMPS* | Stroma | 0.37 | 0.0793 | 0.07 |
| IL6ST | Cancer | 1 | 0.0014 | 0.21 |
| LRBA | Stroma | 1.4 | 0.0008 | 0.22 |
| MAPRE2* | Stoma | 0.48 | 0.0154 | 0.12 |
| MCM6 | Stroma | 0.86 | 0.0044 | 0.16 |
| MELK | Stoma | 0.74 | <0.0001 | 0.46 |
| NAT1* | Cancer | 0.96 | <0.0001 | 0.83 |
| PFKP | Stroma | 0.68 | <0.0001 | 0.53 |
| PLK1* | Stoma | 0.53 | 0.0375 | 0.09 |
| PTP4A2 | Stroma | 1.1 | 0.0009 | 0.21 |
| RABEP1 | Cancer | 1.1 | <0.0001 | 0.44 |
| SCUBE2* | Cancer | 1.2 | <0.0001 | 0.88 |
| SLC39A6 | Cancer | 1.8 | <0.0001 | 0.59 |
| SLC43A3 | Stroma | 0.98 | <0.0001 | 0.40 |
| ST8SIA1 | Cancer | 0.65 | <0.0001 | 0.52 |
| TBC1D9 | Cancer | 1 | <0.0001 | 0.53 |
| TCEAL1 | Cancer | 1.1 | <0.0001 | 0.68 |
| TPBG | Cancer | 0.87 | <0.0001 | 0.57 |
| TRIM29 | Cancer | 1.1 | <0.0001 | 0.66 |
| XBP1 | Stoma | 0.92 | <0.0001 | 0.70 |
| YBX1 | Stoma | 0.63 | 0.0037 | 0.17 |

(*indicates data shown in FIGS. 9A-9D).

TABLE 4
Results from the Cox-regression-survival analysis

| | | Hazard | | | Hazard |
|---|---|---|---|---|---|
| Gene ID | P value | Ratio | Gene ID | P value | Ratio |
| SLC39A6 | 0.012 | 0.83 | XBP1 | 0.281 | 0.88 |
| TPBG | 0.013 | 0.69 | FUT8 | 0.286 | 0.90 |
| TBC1D9 | 0.018 | 0.86 | EVL | 0.298 | 0.88 |
| RABEP1 | 0.024 | 0.76 | CX3CL1 | 0.410 | 0.91 |
| IL6ST | 0.050 | 0.85 | MCM6 | 0.414 | 1.10 |
| ESR1 | 0.058 | 0.90 | GABRP | 0.494 | 0.96 |
| NAT1 | 0.109 | 0.89 | CKS2 | 0.579 | 1.06 |
| MAPRE2 | 0.110 | 0.83 | MELK | 0.601 | 1.07 |
| PTP4A2 | 0.132 | 0.81 | SLC43A3 | 0.675 | 0.94 |
| TCEAL1 | 0.154 | 0.83 | YBX1 | 0.740 | 1.07 |
| GMPS | 0.155 | 0.84 | ATAD2 | 0.807 | 1.05 |
| SCUBE2 | 0.212 | 0.92 | BUB1 | 0.807 | 1.03 |
| LRBA | 0.220 | 0.91 | PFKP | 0.818 | 0.97 |
| ST8SIA1 | 0.229 | 0.84 | PLK1 | 0.878 | 0.97 |
| DSC2 | 0.231 | 0.89 | CENPA | 0.950 | 0.99 |
| GATA3 | 0.263 | 0.92 | TRIM29 | 0.959 | 1.00 |

To relate the results from qPCR measurements of the level of expression of the gene subset (see Table 1) with patient parameters, tumor marker analyses, patient characteristics (e.g., age, menopausal status), tumor properties (e.g., pathology, grade) and clinical outcome (e.g., disease-free and overall survival) were analyzed.

Using the IRB-approved Biorepository and Database of the Hormone Receptor Laboratory, de-identified specimens of primary invasive ductal carcinoma were examined. Tissue-based properties (e.g., pathology of the cancer, grade and size) and encoded patient-related characteristics (e.g., age, race, menopausal status, stage, nodal status, tumor marker status) were utilized to examine the relationships between gene expression results and clinical parameters.

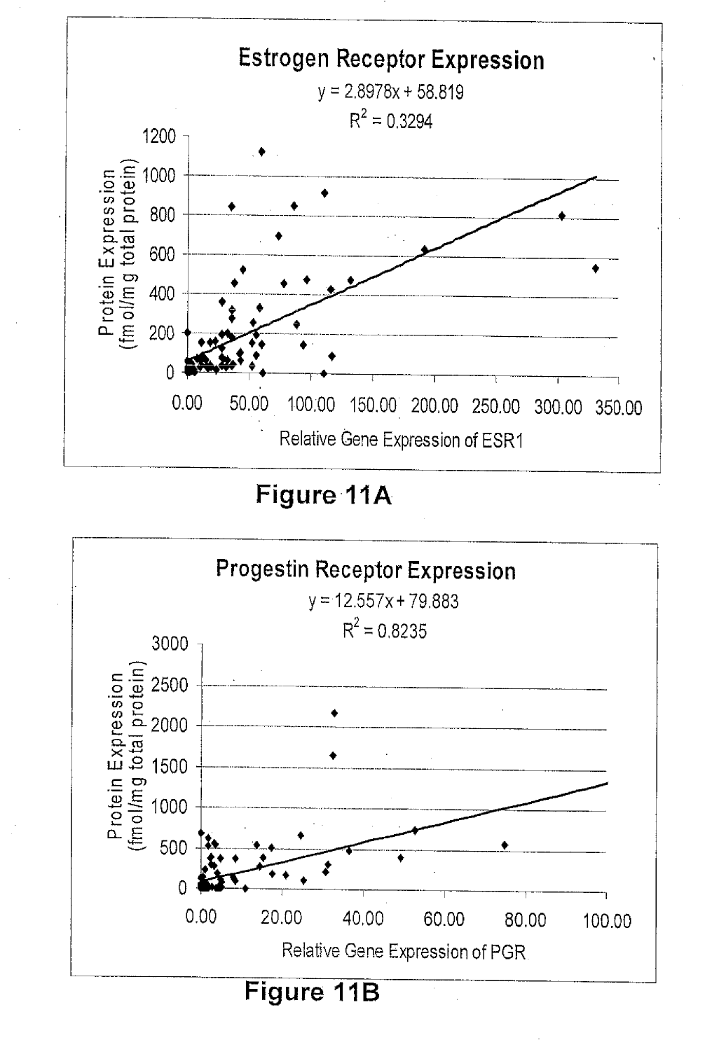

Levels of mRNA expression were analyzed for all 32 genes (Table 1), while receptor protein levels were identified in the Hormone Receptor Laboratory's Database. Comparisons between mRNA expression from an intact tissue section and protein expression from a tissue extract were made in 97 specimens (the 78 outlined in Table 5 plus 19 from an additional study) for estrogen receptor (ER) and progestin receptor (PR) (FIGS. 11A and 11B). The relationship between ER mRNA and protein product levels gave a correlation with r²=0.32, while the correlation between PR mRNA and protein product yielded an r²=0.33, which correlates coefficients from linear regressions made by comparing the mRNA with protein levels. These levels do not correlate for several reasons. Some of the mRNA may either not be translated into a protein product, or the protein may have an unusual turnover rate leading to an accumulation or excessive degradation, depending on the situation in the cell.

TABLE 5
Characteristics of the patient population studied

| Patient Parameters | n |
|---|---|
| Median Age (range) | |
| 56 years (29-89.5) | 78 |
| Median Observation time (range) | |
| 61 months (3-147) | 78 |
| Race | |
| white | 73 |
| black | 5 |
| Histology | |
| Invasive ductal carcinoma | 78 |
| Median Tumor Size (Range) | |
| 29 mm (4-85) | 73 |
| Stage | |
| 1 | 9 |
| 2 | 51 |
| 3 | 9 |
| 4 | 5 |
| unknown | 4 |
| Grade | |
| 1 | 4 |
| 2 | 24 |
| 3 | 30 |
| 4 | 2 |
| unknown | 18 |
| Lymph Node Status | |
| negative | 32 |
| positive | 40 |
| unknown | 6 |
| Recurrence Status | |
| yes | 25 |
| no | 48 |
| never disease-free | 5 |

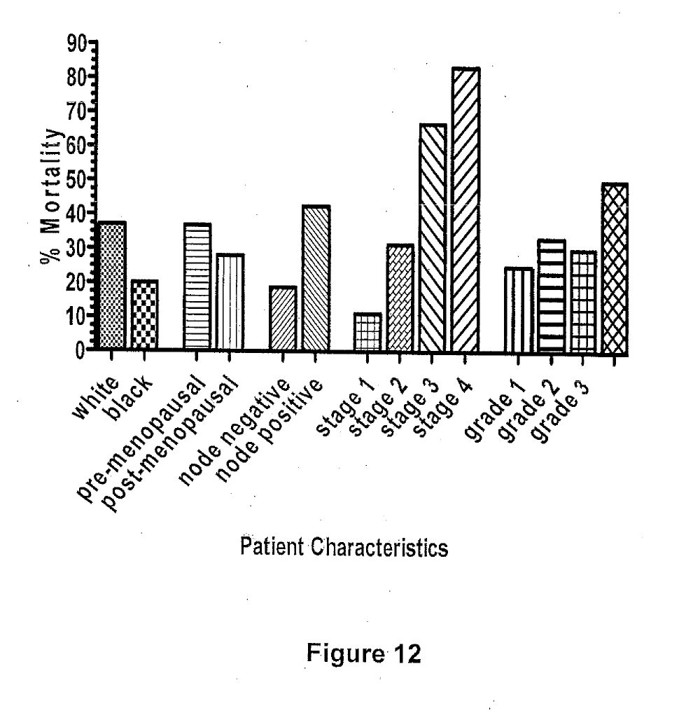

The qPCR data will be correlated with de-identified patient characteristics and clinical data. The characteristics of the study population thus far are described in Table 5. In order to analyze survival with known characteristics of the study population, a percent mortality analysis was performed for each category, including race, menopausal status, lymph node involvement, stage of the cancer and tumor grade (FIG. 12). The percent mortality for patients with clinical stage and grade followed expected outcome, with the exception of race. This may be due to the small sample size of black patients in this population. This can be evaluated as a larger data set is completed.

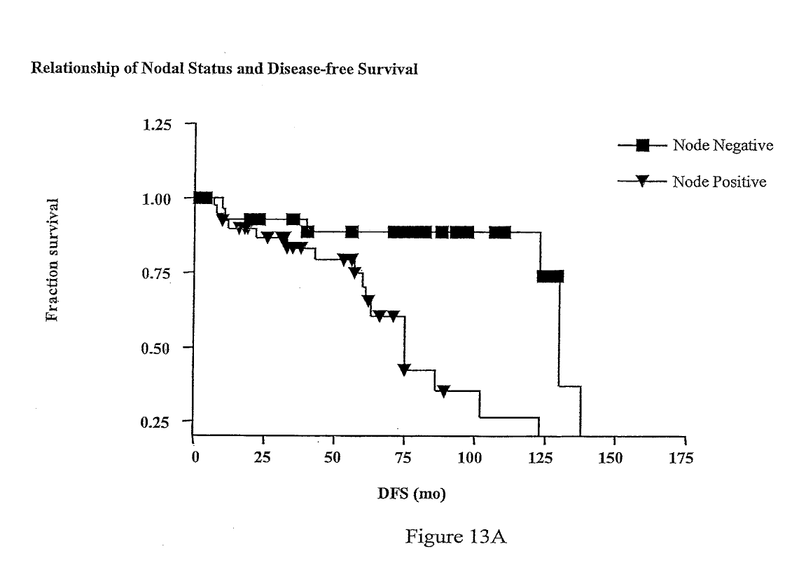

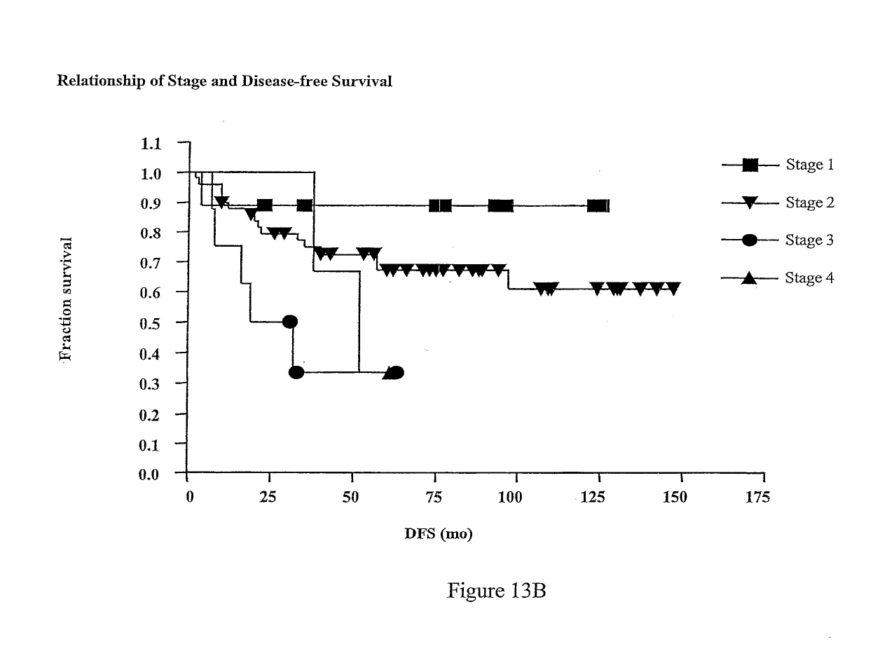

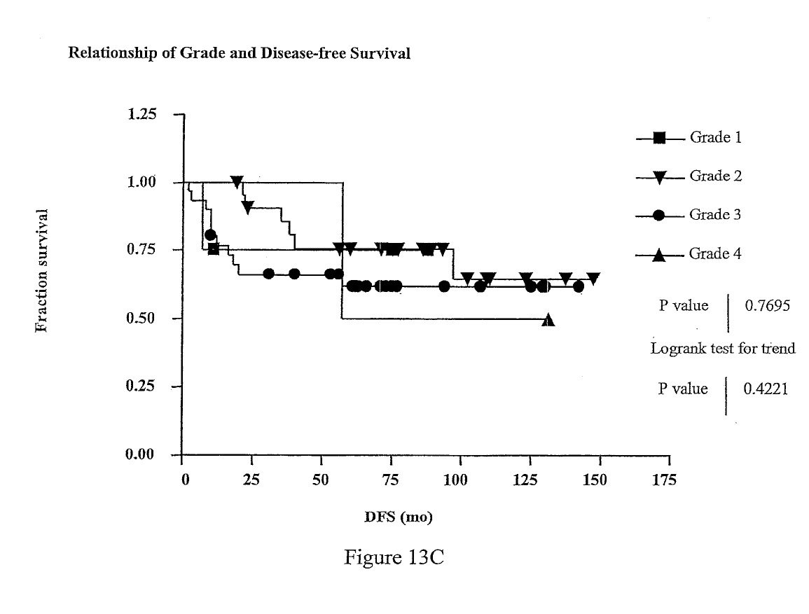

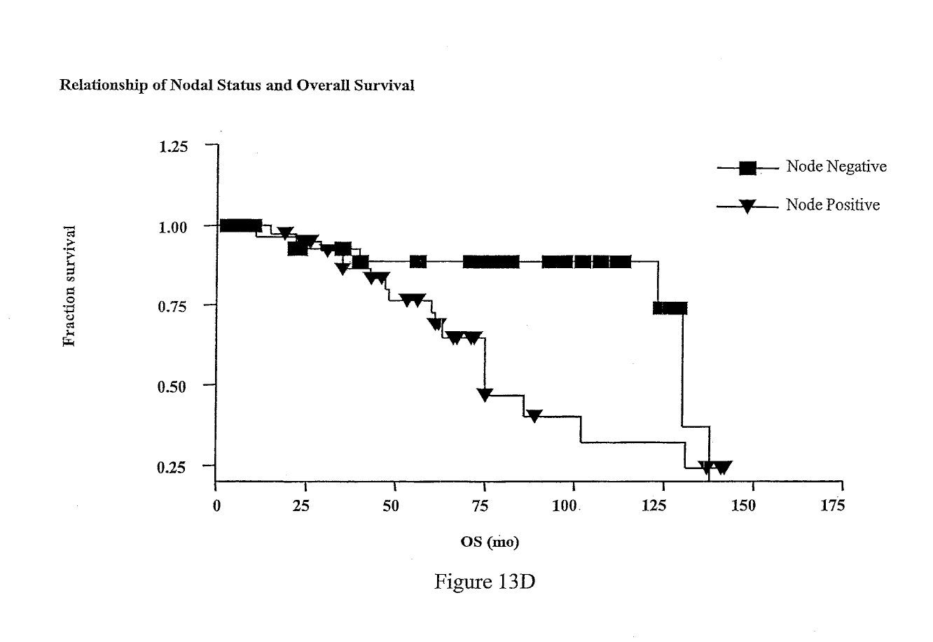

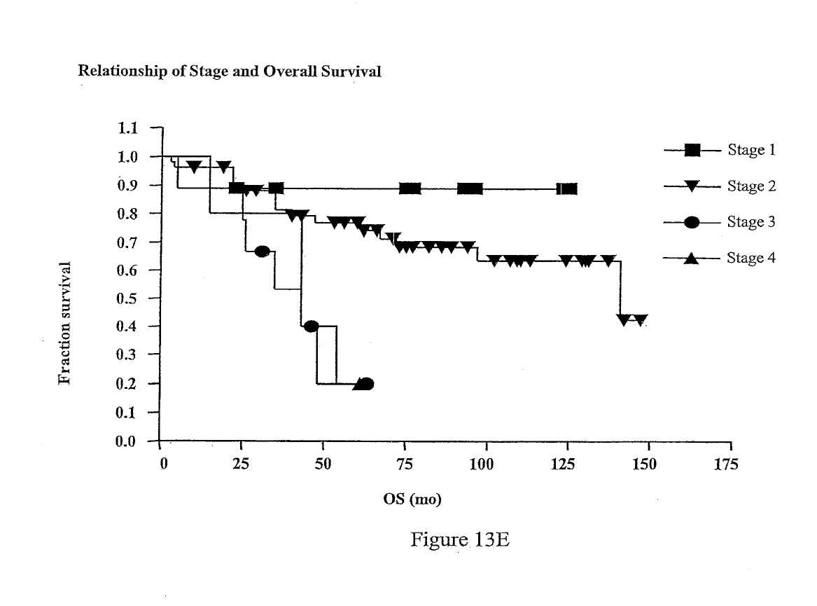

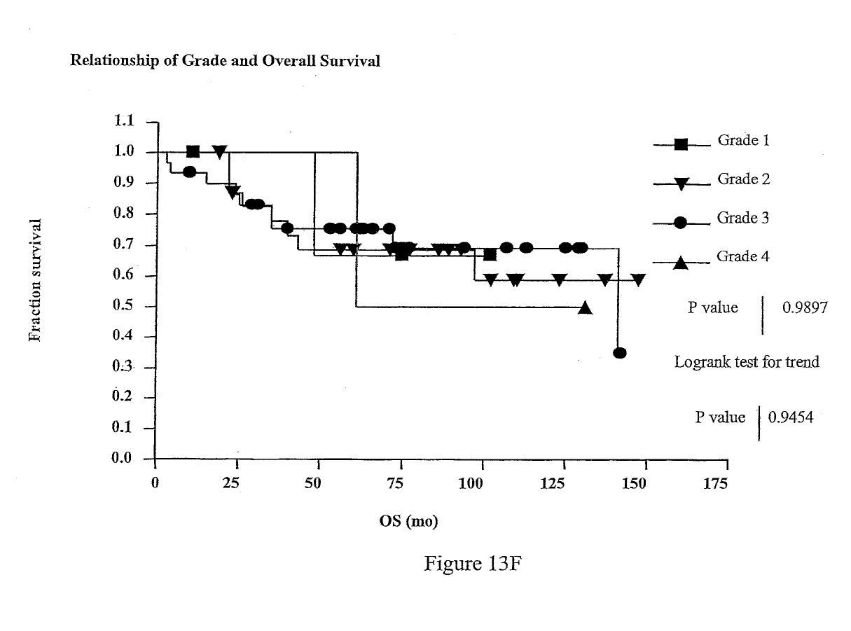

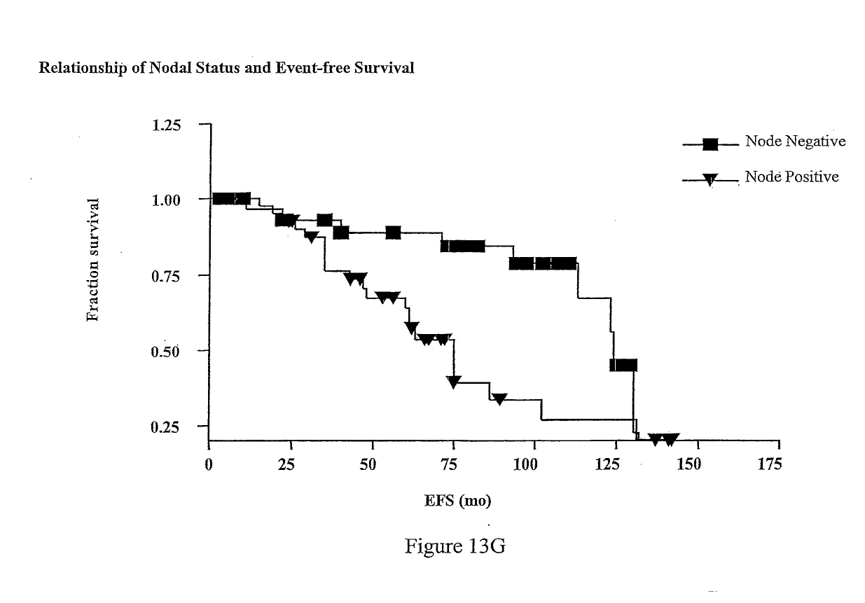

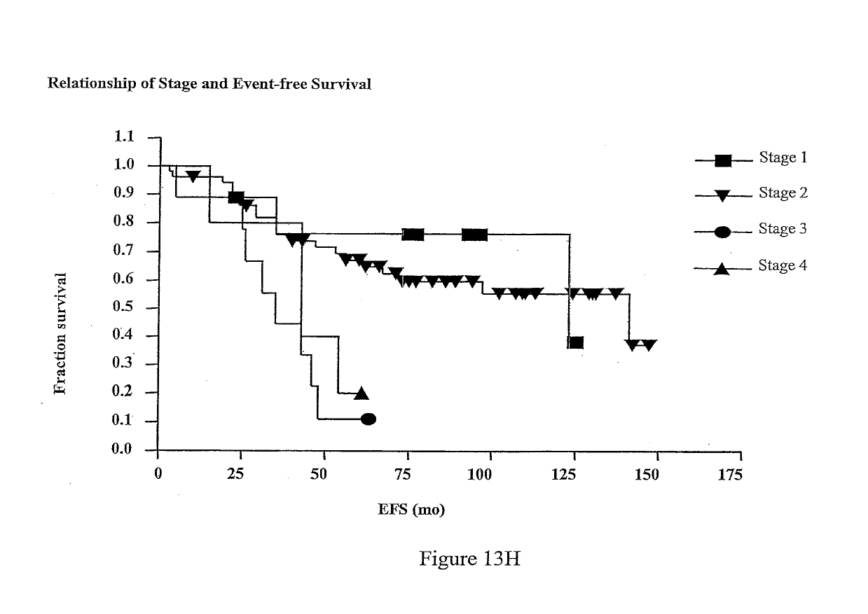

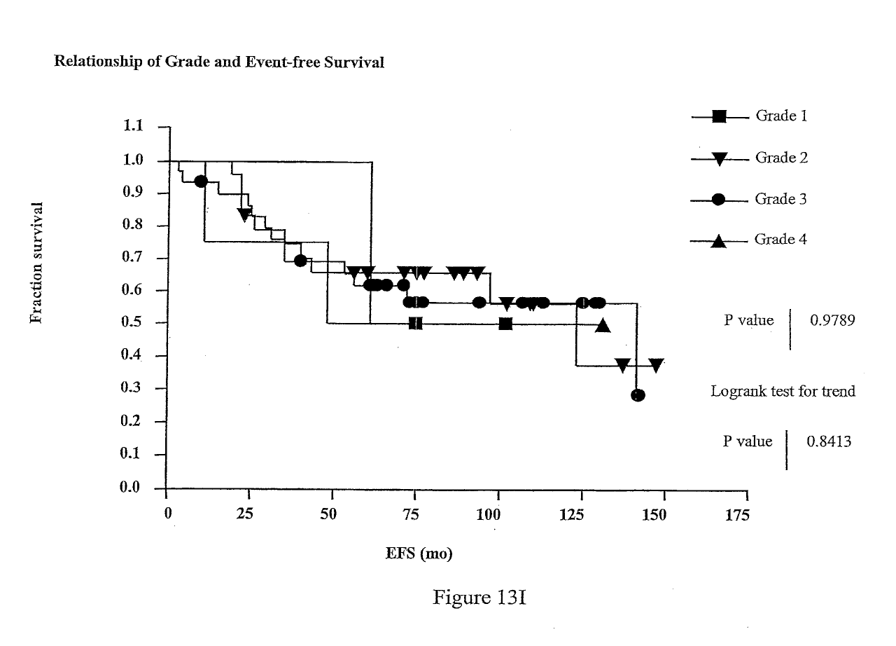

Before gene expression was analyzed for impacting cancer recurrence and survival, known prognostic factors, such as stage, grade and lymph node involvement, were evaluated by Kaplan-Meier survival plots using GraphPad Prism™ software (FIGS. 13A-13I). This software allows a statistical analysis of gene expression and its association with recurrence of the cancer (disease-free survival—DFS), death of the patient due to that cancer (overall survival—OS), and death by any means (event-free survival—EFS). Lymph node involvement, which is considered one of the most important clinical prognostic factors in breast cancer, separated significantly into good prognosis and poor prognosis groups for DFS (P value=0.005), OS (P value=0.012) and EFS (P value=0.017). Stage exhibited significant separation into good and poor prognosis groups for DFS (P value=0.033), OS (P value=0.004) and EFS (P value=0.004), and expected trends in were observed for each stage in all three analyses. Tumor grade did not predict survival. Because the known prognostic factors exhibited expected survival patterns, it appears that an unbiased patient population was sampled.

The expression of each gene was analyzed for associations with the characteristics of each of 78 patients, such as race, menopausal status, stage of disease, tumor grade and nodal involvement, with the use of PARTEK® GENOMICS SUITE™ software (Table 6). Analysis of race, menopausal status, nodal status, ER status and PR status were performed using a standard t-test, while stage, grade and family history were analyzed by ANOVA. The genes shown in Table 6 exhibited P values <0.05.

TABLE 6
Association of gene expression in the carcinoma and stromal subsets
with patient characteristic.

| | |
|---|---|
| Race | no associations |
| Menopausal Status | ATAD2, YBX1, CENPA, PLK1, MELK, PTP4A2, CKS2, GABRP, |
| | TRIM29, ESR1 |
| Family History | ATAD2 |
| Stage | no associations |
| Grade | GMPS, MCM6, PFKP, BUB1, XBP1, SCUBE2, DSC2, EVL |
| Nodal Status | MAPRE2 |
| ER Status | XBP1, FUT8, PFKP, GATA3, SLC43A3, PTP4A2, LRBA, |
| | CX3CL1, MELK, YBX1, ST8SIA1, ESR1, GABRP, NAT1, |
| | RABEP1, EVL, TCEAL1, TBC1D9, SLC39A6, TPBG, SCUBE2 |
| PR Status | XBP1, FUT8, PTP4A2, GATA3, PFKP, CX3CL1, SLC43A3, |
| | MELK, NAT1, EVL, ST8SIA1, ESR1, RABEP1, SLC39A6, |
| | TBC1D9, GABRP, TCEAL1 |

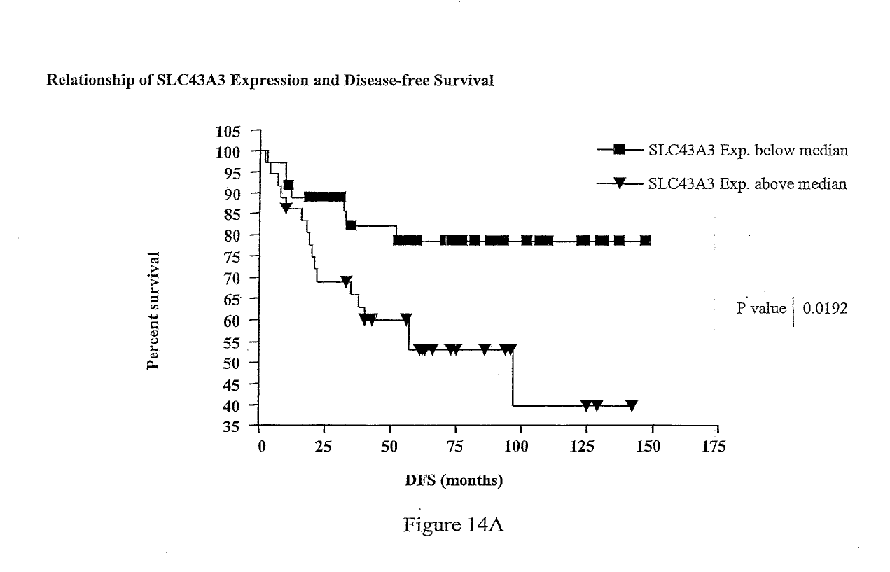

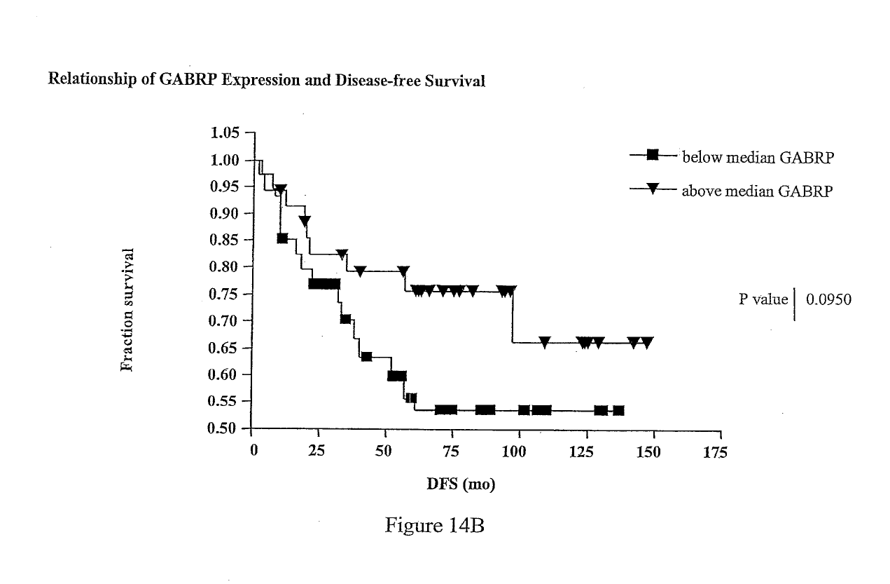

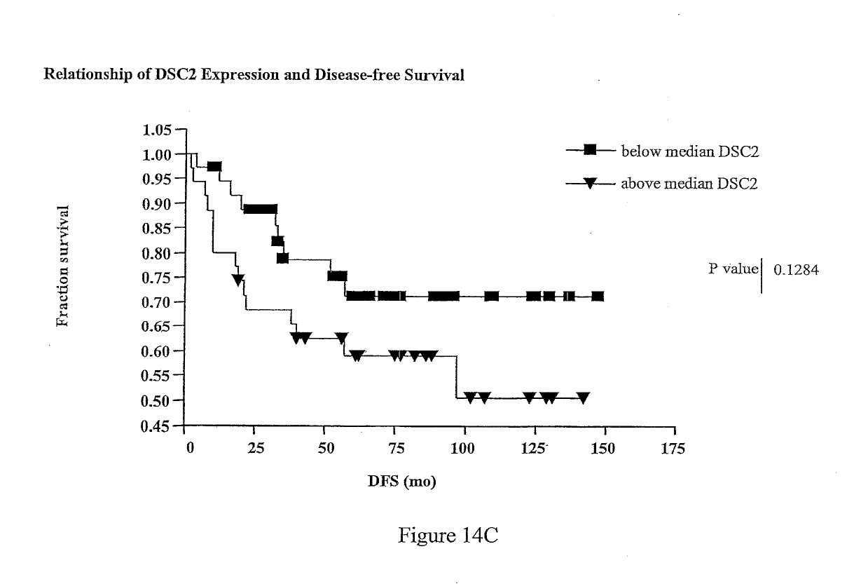

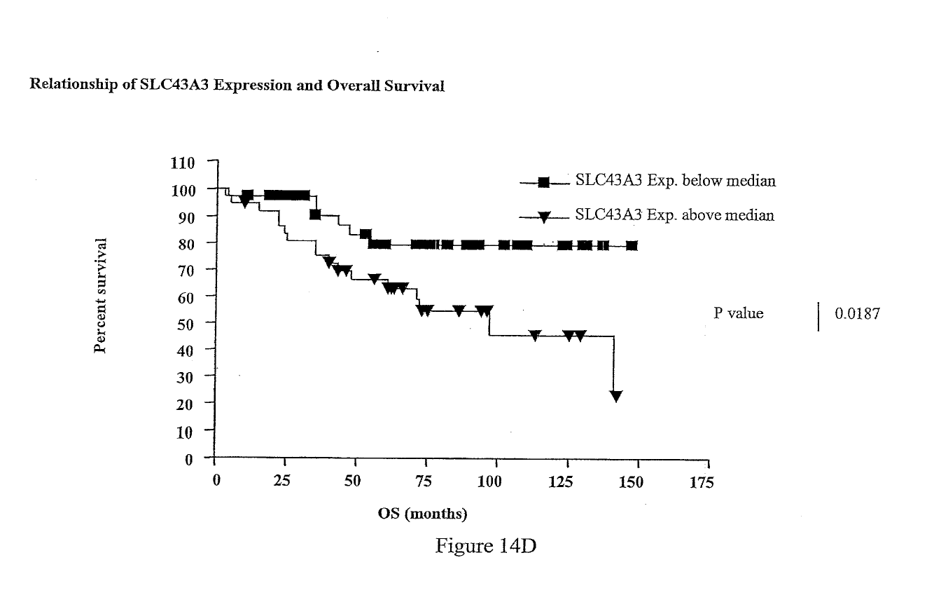

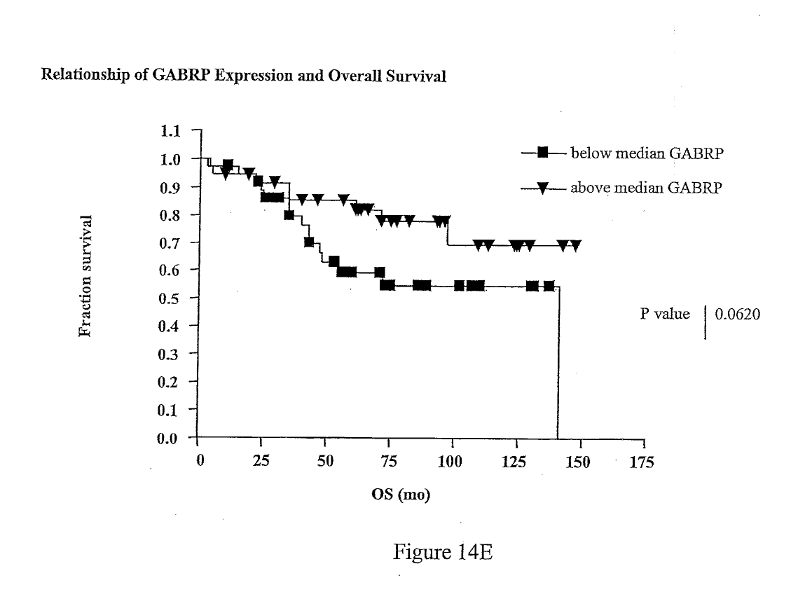

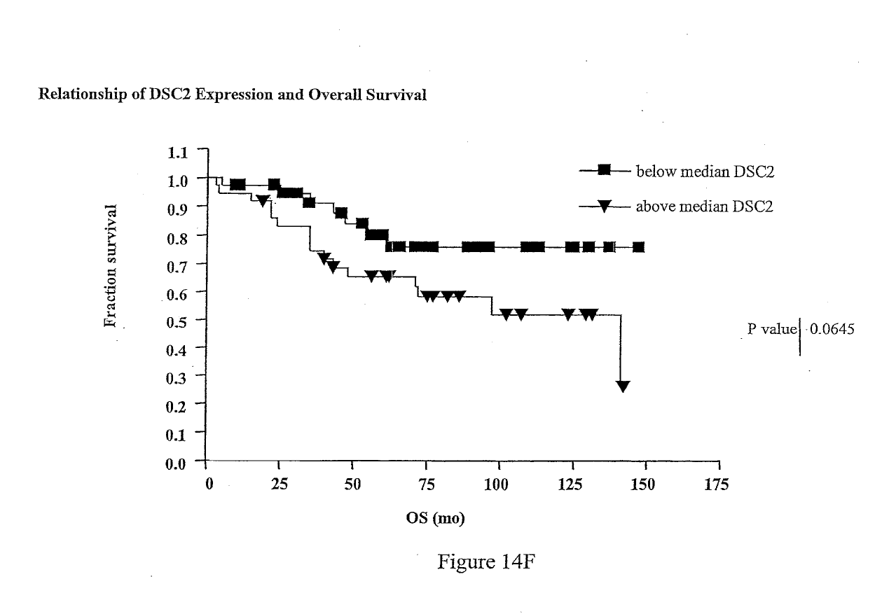

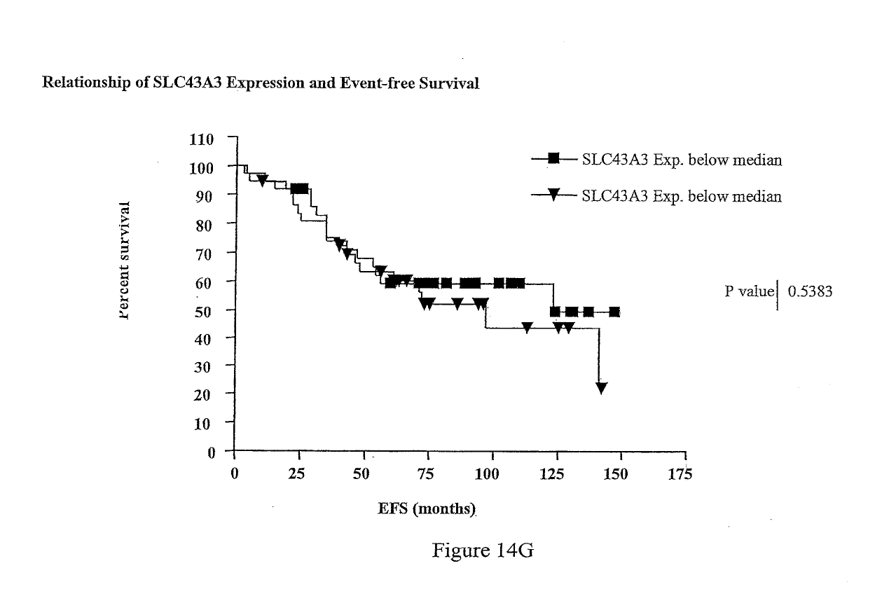

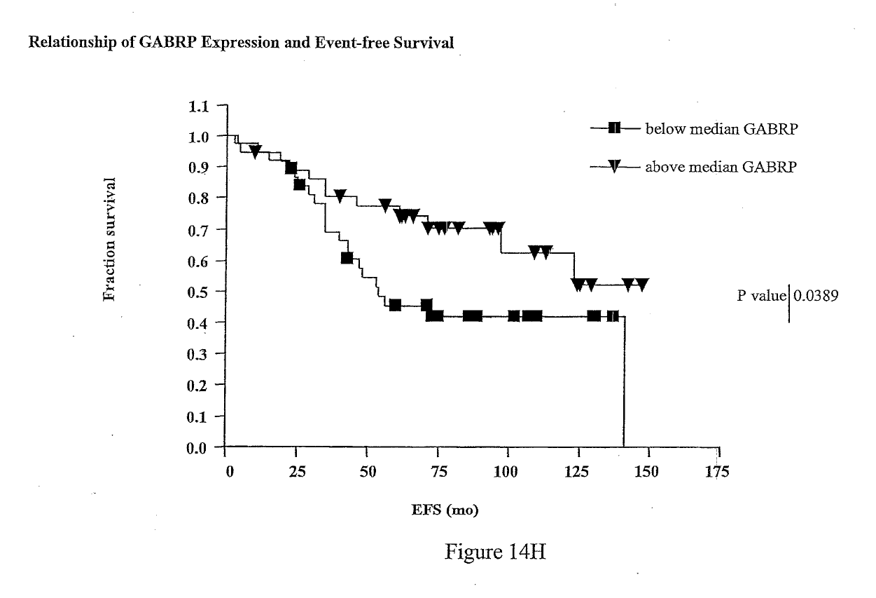

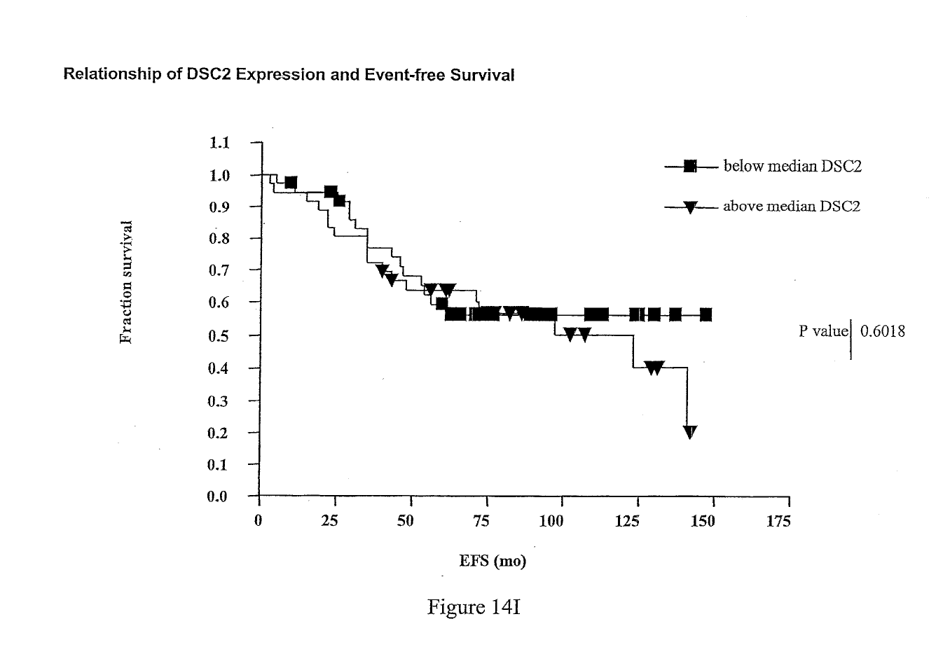

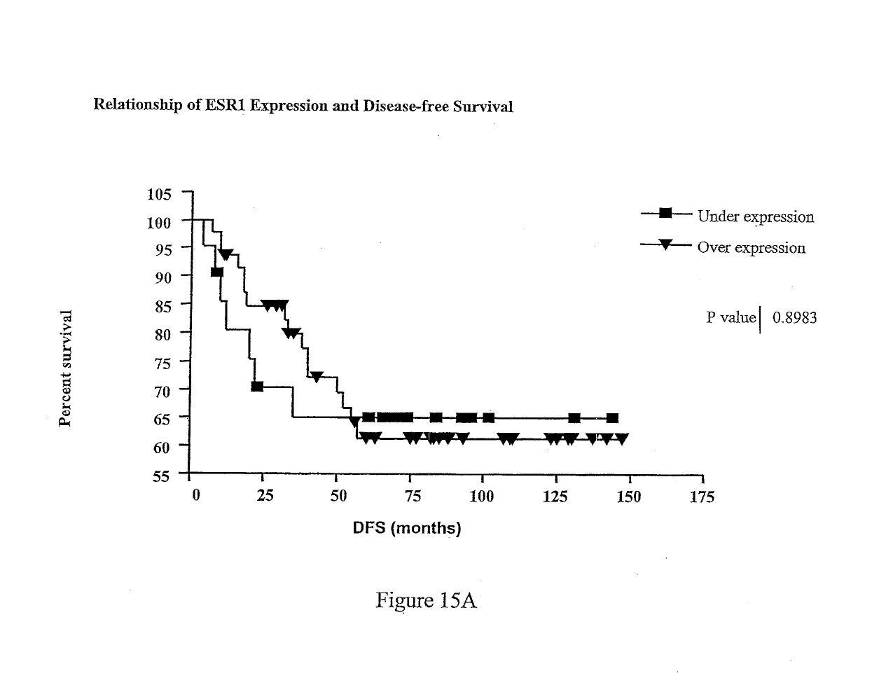

Expression of each gene was then evaluated by Kaplan-Meier analyses using expression above and below median relative expression values to stratify patients (FIGS. 14A-14I, Table 7). Not all of the genes tested showed correlations with recurrence and survival, but some appear to indicate trends which separate patients into groups. Of the 32 genes evaluated in the gene subsets, 8 genes (CENPA, DSC2, GABRP, GATA3, MAPRE2, RABEP1, SCUBE2, SLC43A3) appear to be moderately associated with either recurrence or overall survival with a P value less than 0.20. Only one of the genes (SLC43A3) individually predicted recurrence or overall survival with a P value less than 0.05. The Hazard Ratios for each gene are shown (Table 7), but it should be noted that these are only representative of the gene once defined significant. Since 247 patients were evaluated in a previous study, there may be greater statistical significance within the larger sample population. Similar evaluations using the LCM-procured pure cell populations can also be performed, although with a smaller sample size. These expression studies were performed by analyzing expression of each gene individually. However, it is likely that there will be a much greater power of prediction when the genes are analyzed collectively.

Further statistical analysis was done to assess the association of gene expression in the carcinoma and stromal subsets with patient characteristic. Two-sample t-tests were performed using PARTEK® GENOMICS SUITE™ software. Genes were identified as significant using a p-value of 0.05. A mean gene expression was calculated for each group, e.g., pre-menopausal and post-menopausal. Those mean values were converted to a fold change in expression. The difference in fold change between groups was calculated and genes were reported which had at least a 2-fold change in expression (Table 8).

TABLE 7
Results from Kaplan Meier analylses of genes for
disease-free, overall and event-free survival.

| | Disease-free | | Overall | | Event-free | |
|---|---|---|---|---|---|---|
| | Survival | | Survival | | Survival | |
| | | Hazard | | Hazard | | Hazard |
| Gene ID | P value | Ratio | P value | Ratio | P value | Ratio |
| ATAD2 | 0.757 | 0.88 | 0.960 | 0.98 | 0.873 | 0.95 |
| BUB1 | 0.704 | 1.17 | 0.824 | 1.10 | 0.867 | 0.94 |
| CENPA | 0.254 | 0.62 | 0.133 | 0.53 | 0.572 | 0.83 |
| CKS2 | 0.808 | 1.10 | 0.914 | 1.05 | 0.576 | 1.21 |
| CX3CL1 | 0.352 | 1.46 | 0.899 | 1.05 | 0.665 | 1.16 |
| DSC2* | 0.128 | 0.53 | 0.065 | 0.45 | 0.602 | 0.83 |
| ESR1 | 0.900 | 1.05 | 0.945 | 0.97 | 0.308 | 0.70 |
| EVL | 0.842 | 0.92 | 0.926 | 0.96 | 0.491 | 0.79 |
| FUT8 | 0.702 | 1.17 | 0.816 | 1.10 | 0.478 | 1.27 |
| GABRP* | 0.095 | 1.85 | 0.062 | 2.20 | 0.039 | 2.10 |
| GATA3 | 0.392 | 0.71 | 0.156 | 0.55 | 0.108 | 0.57 |
| GMPS | 0.729 | 0.71 | 0.813 | 0.55 | 0.108 | 0.57 |
| IL6ST | 0.693 | 1.17 | 0.861 | 1.08 | 0.491 | 1.27 |
| LRBA | 0.945 | 0.97 | 0.828 | 0.91 | 0.555 | 0.82 |
| MAPRE2 | 0.205 | 0.60 | 0.140 | 0.54 | 0.567 | 0.82 |
| MCM6 | 0.700 | 1.17 | 0.752 | 1.14 | 0.986 | 1.01 |
| MELK | 0.550 | 0.78 | 0.787 | 0.89 | 0.670 | 1.16 |
| NAT1 | 0.834 | 1.09 | 0.949 | 0.97 | 0.482 | 0.78 |
| PFKP | 0.542 | 0.78 | 0.688 | 0.85 | 0.754 | 1.12 |
| PLK1 | 0.248 | 0.62 | 0.202 | 0.58 | 0.186 | 0.63 |
| PTP4A2 | 0.631 | 0.82 | 0.610 | 0.81 | 0.227 | 0.66 |
| RABEP1 | 0.178 | 1.73 | 0.201 | 1.69 | 0.197 | 1.56 |
| SCUBE2 | 0.105 | 1.95 | 0.223 | 1.67 | 0.752 | 1.12 |
| SLC39A6 | 0.214 | 1.66 | 0.238 | 1.63 | 0.409 | 1.33 |
| SLC43A3* | 0.019 | 0.37 | 0.019 | 0.35 | 0.538 | 0.81 |
| ST8SIA1 | 0.587 | 0.81 | 0.858 | 0.93 | 0.597 | 1.21 |
| TBC1D9 | 0.696 | 1.17 | 0.807 | 1.11 | 0.474 | 1.28 |
| TCEAL1 | 0.821 | 0.91 | 0.666 | 0.84 | 0.156 | 0.61 |
| TPBG | 0.921 | 1.04 | 0.985 | 0.99 | 0.774 | 0.91 |
| TRIM29 | 0.914 | 1.05 | 0.437 | 1.37 | 0.083 | 1.83 |
| XBP1 | 0.682 | 1.18 | 0.459 | 1.36 | 0.975 | 0.99 |
| YBX1 | 0.771 | 1.13 | 0.763 | 0.89 | 0.377 | 1.45 |

(*indicates data shown in FIGS. 14A-14I).

TABLE 8
Association of gene expression in the carcinoma and stromal subsets with
patient characteristics

| | | | |
|---|---|---|---|
| Race | white | n = 73 | no associations |
| | black | n = 5 | no associations |
| Menopausal | pre | n = 19 | GABRP, ESR1 |
| Status | | | |
| | post | n = 23 | no associations |
| Family History | no | n = 23 | no associations |
| | yes | n = 15 | no associations |
| Stage | 1 | n = 9 | no associations |
| | 2 | n = 51 | no associations |
| | 3 | n = 9 | no associations |
| | 4 | n = 5 | no associations |
| Grade | 1 | n = 4 | MCM6, PFKP, BUB1, XBP1 |
| | 2 | n = 24 | EVL |
| | 3&4 | n = 32 | GMPS, SCUBE2, DSC2 |
| Nodal Status | neg | n = 32 | no associations |
| | pos | n = 40 | no associations |
| ER Status | neg | n = 26 | XBP1, MELK, ST8SIA1, |
| | | | GABRP |
| | pos | n = 52 | FUT8, CX3CL1, ESR1, |
| | | | NAT1, RABEP1, EVL, |
| | | | TCEAL1, TBC1D9, SLC39A6, |
| | | | SCUBE2 |
| PR Status | neg | n = 27 | GABRP, MELK, ST8SIA1 |
| | pos | n = 51 | XBP1, FUT8, PTP4A2, |
| | | | SLC39A6, TBC1D9, NAT1, |
| | | | EVL, ESR1, RABEP1 |

Genes shown are upregulated for that characteristic, having at least a 2-fold change between groups and a P value <0.05.

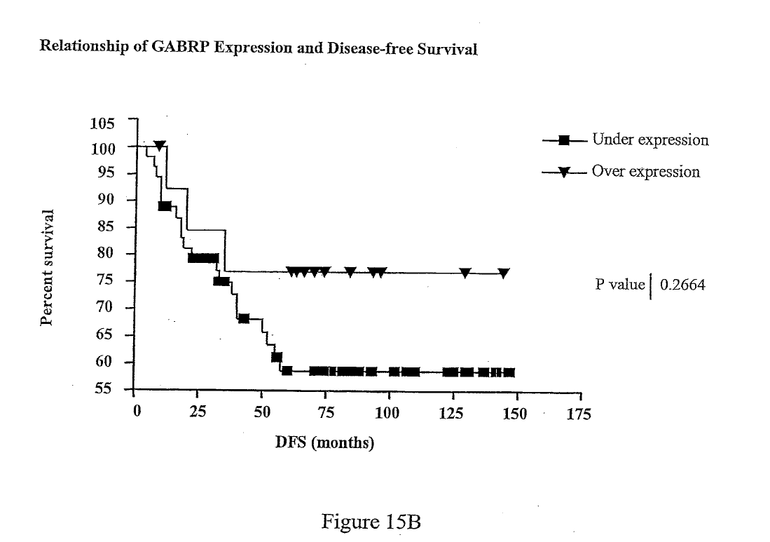

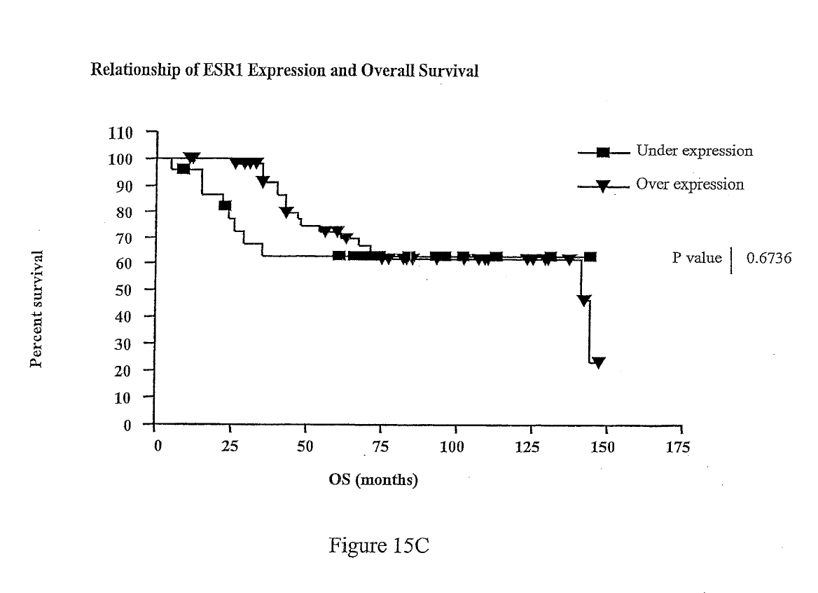

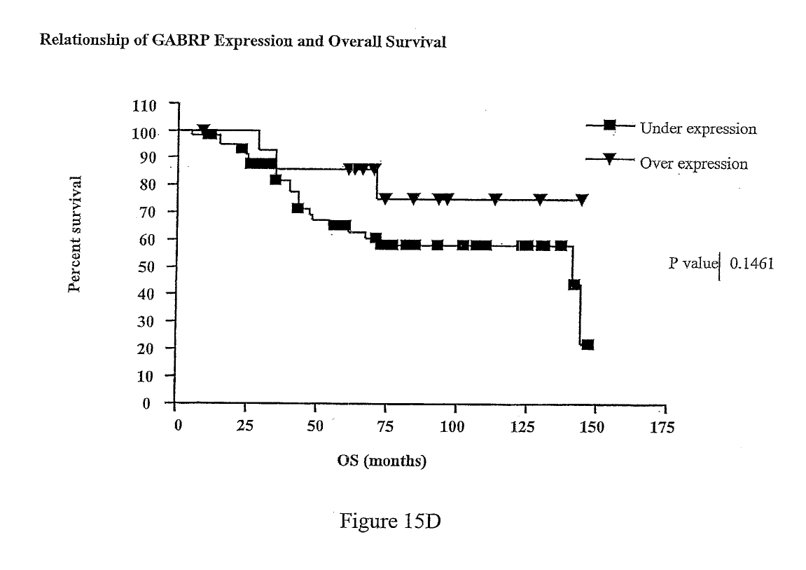

Because results indicated bimodal distribution in the expression of Hs.208124 (ESR1) and Hs.26225 (GABRP) (FIGS. 8B and 8C), those groups with lower gene expression and higher gene expression were also investigated by Kaplan-Meier analysis using a relative gene expression cut-off of 2 for ESR1 and 64 for GABRP (FIGS. 15A-15D). These alternative groupings did not improve the Kaplan-Meier survival analyses of ESR1 or GABRP, and, in fact, the curve separation for GABRP was less statistically significant than using the median expression value (DFS: 0.26 compared to 0.10, OS: 0.15 compared to 0.06).

Another method of survival analysis was performed using the Cox Regression tool within PARTEK® GENOMICS SUITE™ (GeneChip-Compatible: Predicting Clinical Outcome of Cancer Patients—Prognostic Classification & Survival Analysis Using Partek. Affymetrix Web Event. Mar. 29, 2006). The main difference is that a Cox Regression analyzes continuous variables, and does not require separation into groups (e.g., above median, below median) for analysis. This method yielded 4 genes with P values <0.05 (SLC39A6, TPBG, TBC1D9, RABEP1) (Table 3). Because the expression of these genes was statistically significant with this method, different cut-off points (other than the median expression values) may be tried in the Kaplan-Meier analyses to obtain more significant separation.

In order to elucidate a clinically relevant molecular signature from the gene expression data obtained, PARTEK® GENOMICS SUITE™ software is being utilized (Downey T., Methods Enzymol 411:256-270 (2006)). This software package is a comprehensive system of advanced statistics and data visualization specifically designed to extract biological information from large amounts of expression data. By importing relative gene expression data, the software develops a best fitting algorithm for a particular characteristic (i.e., breast cancer recurrence, death due to breast cancer) This algorithm can then be used to predict that particular characteristic in additional samples based on their relative gene expression data. The software will runs a large number of combinations and permutations of genes to develop the most statistically significant algorithm, or molecular signature. These signatures undergo 1-level cross validation by removing 10% of the data 10 times.

Using the log₂ expression data from all 32 genes analyzed in whole tissue sections, the patients were randomly placed into Training and Test Sets at a ratio of about 50% to about 50%, respectively. The Training and Test Set were divided at a ratio of about 60% to about 40%, and will use this in future analyses. In other words, the patient population will be randomly divided so that about 60% of the patients will be in the training set and the remaining about 40% will be the test set. Using the Training Set data to predict disease recurrence, the following types of models were analyzed with 1 to 32 genes and any combination thereof: K-nearest neighbor, linear discriminant (equal and proportional prior probability), quadratic discriminant (equal and proportional prior probability), nearest centroid (equal and proportional prior probability). The top 5 models during cross validation were stored and analyzed using the Test Set data (Tables 9-14).

Data from an additional 7 specimens have been collected and another 6 have been prepared for qPCR. A complete analysis will be repeated once the data set exceeds the statistical requirement, estimated to be more than 100 patient samples. A similar analysis may be performed on the LCM-procured cells even though the sample size will be much smaller.

TABLE 9
Top 5 models after 1-level cross validation with PARTEK ® GENOMICS
SUITE ™ predicting recurrence.

| | |
|---|---|
| Model 1 | 21 variables, K-Nearest Neighbor with Euclidean distance |
| | measure and 1 neighbor |
| Model 2 | 20 variables, K-Nearest Neighbor with Euclidean distance |
| | measure and 1 neighbor |
| Model 3 | 28 variables, Linear Discriminant Analysis with Equal Prior |
| | Probability |
| Model 4 | 24 variables, Quadratic Discriminant Analysis with Proportional |
| | Prior Probability |
| Model 5 | 28 variables, Quadratic Discriminant Analysis with Proportional |
| | Prior Probability |

TABLE 10
Genes of Model 1

| UniGene Identifier | Gene Description |
|---|---|
| Hs.208124 | ESR1 |
| Hs.26225 | GABRP |
| Hs.480819 | TBC1D9 |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.1594 | CENPA |
| Hs.184339 | MELK |
| Hs.26010 | PFKP |
| Hs.592049 | PLK1 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.99962 | SLC43A3 |

TABLE 11
Genes of Model 2

| UniGene Identifier | Gene Description |
|---|---|
| Hs.208124 | ESR1 |
| Hs.26225 | GABRP |
| Hs.480819 | TBC1D9 |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.184339 | MELK |
| Hs.26010 | PFKP |
| Hs.592049 | PLK1 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.99962 | SLC43A3 |

TABLE 12
Genes of Model 3

| UniGene Identifier | Gene Description |
|---|---|
| Hs.125867 | EVL |
| Hs.208124 | ESR1 |
| Hs.26225 | GABRP |
| Hs.408614 | ST8SIA1 |
| Hs.480819 | TBC1D9 |
| Hs.504115 | TRIM29 |
| Hs.523468 | SCUBE2 |
| Hs.532082 | IL6ST |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.1594 | CENPA |
| Hs.184339 | MELK |
| Hs.26010 | PFKP |
| Hs.592049 | PLK1 |
| Hs.370834 | ATAD2 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.532824 | MAPRE2 |
| Hs.99962 | SLC43A3 |

TABLE 13
Genes of Model 4

| UniGene Identifier | Gene Description |
|---|---|
| Hs.208124 | ESR1 |
| Hs.26225 | GABRP |
| Hs.480819 | TBC1D9 |
| Hs.523468 | SCUBE2 |
| Hs.532082 | IL6ST |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.1594 | CENPA |
| Hs.184339 | MELK |
| Hs.26010 | PFKP |
| Hs.592049 | PLK1 |
| Hs.370834 | ATAD2 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.99962 | SLC43A3 |

TABLE 14
Genes of Model 5

| UniGene Identifier | Gene Description |
|---|---|
| Hs.125867 | EVL |
| Hs.208124 | ESR1 |
| Hs.26225 | GABRP |
| Hs.408614 | ST8SIA1 |
| Hs.480819 | TBC1D9 |
| Hs.504115 | TRIM29 |
| Hs.523468 | SCUBE2 |
| Hs.532082 | IL6ST |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.1594 | CENPA |
| Hs.184339 | MELK |
| Hs.26010 | PFKP |
| Hs.592049 | PLK1 |
| Hs.370834 | ATAD2 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.532824 | MAPRE2 |
| Hs.99962 | SLC43A3 |

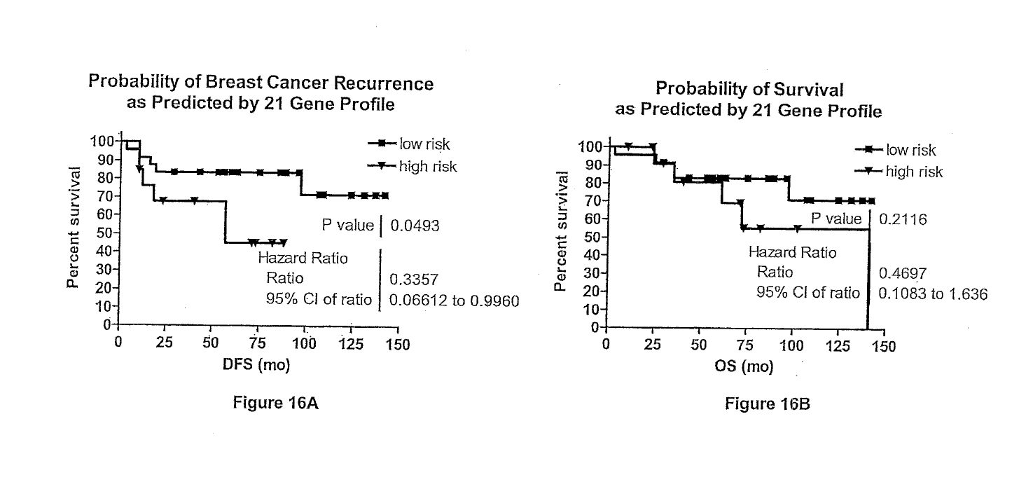

The model that best predicted disease recurrence is “K-nearest neighbor with Euclidean distance measure and 1 neighbor” using 21 genes (Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3)) (Tables 9 and 10). This model was then deployed against the 37 patient Test Set population, and Kaplan-Meier analyses were performed (FIGS. 16A and 16B). The 21 gene model predicted disease-free survival with a P value of 0.049 and a hazard ratio of about 0.34, indicating that a gene expression profile fitting the low risk group predicts approximately a 3-fold less probability of cancer recurrence. The risk groups predicted by the model were also analyzed for overall survival of the patients yielding a P value of 0.212 and a hazard ratio of about 0.47.

Additional patient characteristics (e.g., menopausal status, race, family history, tumor grade, stage of disease, lymph node status, estrogen receptor status, progestin receptor status) can be converted to numerical values and utilized in developing the best fitting algorithm, which allows the signature to incorporate all available information, both standard prognostic factors and gene expression combined, to most accurately predict a patient's clinical outcome. Additional multivariate analyses are being performed in order to best analyze all available data.

The methods described herein can identify expression of genes listed in Tables 1-36.

TABLE 15
Genes of the carcinoma subset

| UniGene Identifier | Gene Description |
|---|---|
| Hs.125867 | EVL |
| Hs.591847 | NAT1 |
| Hs.208124 | ESR1 |
| Hs.26225 | GABRP |
| Hs.408614 | ST8SIA1 |
| Hs.480819 | TBC1D9 |
| Hs.504115 | TRIM2 |
| Hs.523468 | SCUBE2 |
| Hs.532082 | IL6ST |
| Hs.592121 | RABEP1 |
| Hs.79136 | SLC39A6 |
| Hs.82128 | TPBG |
| Hs.95243 | TCEAL1 |
| Hs.95612 | DSC2 |

TABLE 16
Genes of the stromal cell subset

| UniGene Identifier | Gene Description |
|---|---|
| Hs.654961 | FUT8 |
| Hs.1594 | CENPA |
| Hs.184339 | MELK |
| Hs.26010 | PFKP |
| Hs.592049 | PLK1 |
| Hs.370834 | ATAD2 |
| Hs.437638 | XBP1 |
| Hs.444118 | MCM6 |
| Hs.469649 | BUB1 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.532824 | MAPRE2 |
| Hs.591314 | GMPS |
| Hs.83758 | CKS2 |
| Hs.99962 | SLC43A3 |

TABLE 17

| UniGene Identifier | Gene Description |
|---|---|
| Hs.208124 | ESR1 |
| Hs.26225 | GABRP |
| Hs.480819 | TBC1D9 |
| Hs.592121 | RABEP1 |
| Hs.79136 | SLC39A6 |
| Hs.82128 | TPBG |
| Hs.95243 | TCEAL1 |
| Hs.95612 | DSC2 |
| Hs.654961 | FUT8 |
| Hs.1594 | CENPA |
| Hs.184339 | MELK |
| Hs.26010 | PFKP |
| Hs.592049 | PLK1 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.99962 | SLC43A3 |

TABLE 18

| UniGene Identifier | Gene Description |
|---|---|
| Hs.208124 | ESR1 |
| Hs.26225 | GABRP |
| Hs.480819 | TBC1D9 |
| Hs.592121 | RABEP1 |
| Hs.79136 | SLC39A6 |
| Hs.82128 | TPBG |
| Hs.95243 | TCEAL1 |
| Hs.95612 | DSC2 |

TABLE 19

| UniGene Identifier | Gene Description |
|---|---|
| Hs.654961 | FUT8 |
| Hs.1594 | CENPA |
| Hs.184339 | MELK |
| Hs.26010 | PFKP |
| Hs.592049 | PLK1 |
| Hs.437638 | XBP1 |
| Hs.444118 | MCM6 |
| Hs.470477 | PTP4A2 |
| Hs.473583 | YBX1 |
| Hs.480938 | LRBA |
| Hs.524134 | GATA3 |
| Hs.531668 | CX3CL1 |
| Hs.99962 | SLC43A3 |

TABLE 20
Genes with a P value less than or equal to 0.05 from Table 4.

| UniGene Identifier | Gene Description |
|---|---|
| Hs.480819 | TBC1D9 |
| Hs.532082 | IL6ST |
| Hs.592121 | RABEP1 |
| Hs.79136 | SLC39A6 |
| Hs.82128 | TPBG |

TABLE 21
Genes with a P value less than 0.05 from Table 4.

| UniGene Identifier | Gene Description |
|---|---|
| Hs.480819 | TBC1D9 |
| Hs.592121 | RABEP1 |
| Hs.79136 | SLC39A6 |
| Hs.82128 | TPBG |

TABLE 22
Genes with a P value less than 0.02 from Table 4.

| UniGene Identifier | Gene Description |
|---|---|
| Hs.480819 | TBC1D9 |
| Hs.79136 | SLC39A6 |
| Hs.82128 | TPBG |

TABLE 23

| UniGene Identifier | Gene Description |
|---|---|
| Hs.26225 | GABRP |
| Hs.523468 | SCUBE2 |
| Hs.592121 | RABEP1 |
| Hs.95612 | DSC2 |
| Hs.1594 | CENPA |
| Hs.524134 | GATA3 |
| Hs.532824 | MAPRE2 |
| Hs.99962 | SLC43A3 |

TABLE 24
Genes identified as correlating best with microarray data shown in
FIG. 10A-10C.

| UniGene Identifier | Gene Description |
|---|---|
| Hs.591847 | NAT1 |
| Hs.208124 | ESR1 |
| Hs.523468 | SCUBE2 |

TABLE 25

| UniGene Identifier | Gene Description |
|---|---|
| Hs.125867 | EVL |
| Hs.591847 | NAT1 |
| Hs.208124 | ESR1 |
| Hs.26225 | GABRP |
| Hs.408614 | ST8SIA1 |
| Hs.480819 | TBC1D9 |
| Hs.523468 | SCUBE2 |
| Hs.592121 | RABEP1 |
| Hs.79136 | SLC39A6 |
| Hs.82128 | TPBG |
| Hs.95243 | TCEAL1 |
| Hs.654961 | FUT8 |
| Hs.184339 | MELK |
| Hs.26010 | PFKP |
| Hs.437638 | XBP1 |
| Hs.470477 | PTP4A2 |
| Hs.473583 | YBX |
| Hs.480938 | LRBA |
| Hs.524134 | GATA3 |
| Hs.531668 | CX3CL1 |
| Hs.99962 | SLC43A3 |

TABLE 26
Genes associated with estrogen receptor positive breast tissue

| UniGene Identifier | Gene Description |
|---|---|
| Hs.125867 | EVL |
| Hs.591847 | NAT1 |
| Hs.208124 | ESR1 |
| Hs.480819 | TBC1D9 |
| Hs.523468 | SCUBE2 |
| Hs.592121 | RABEP1 |
| Hs.79136 | SLC39A6 |
| Hs.95243 | TCEAL1 |
| Hs.654961 | FUT8 |
| Hs.531668 | CX3CL1 |

TABLE 27
Genes associated with estrogen receptor negative breast tissue

| UniGene Identifier | Gene Description |
|---|---|
| Hs.26225 | GABRP |
| Hs.408614 | ST8SIA1 |
| Hs.184339 | MELK |
| Hs.437638 | XBP1 |

TABLE 28

| UniGene Identifier | Gene Description |
|---|---|
| Hs.125867 | EVL |
| Hs.591847 | NAT1 |
| Hs.208124 | ESR1 |
| Hs.26225 | GABRP |
| Hs.408614 | ST8SIA1 |
| Hs.480819 | TBC1D9 |
| Hs.592121 | RABEP1 |
| Hs.79136 | SLC39A6 |
| Hs.95243 | TCEAL1 |
| Hs.654961 | FUT8 |
| Hs.184339 | MELK |
| Hs.26010 | PFKP |
| Hs.437638 | XBP1 |
| Hs.470477 | PTP4A2 |
| Hs.524134 | GATA3 |
| Hs.531668 | CX3CL1 |
| Hs.99962 | SLC43A3 |

TABLE 29
Genes associated with progestin-receptor positive breast tissue

| UniGene Identifier | Gene Description |
|---|---|
| Hs.125867 | EVL |
| Hs.591847 | NAT1 |
| Hs.208124 | ESR1 |
| Hs.480819 | TBC1D9 |
| Hs.592121 | RABEP1 |
| Hs.79136 | SLC39A6 |
| Hs.654961 | FUT8 |
| Hs.437638 | XBP1 |
| Hs.470477 | PTP4A2 |

TABLE 30
Genes associated with progestin receptor positive breast tissue

| UniGene Identifier | Gene Description |
|---|---|
| Hs.26225 | GABRP |
| Hs.408614 | ST8SIA1 |
| Hs.184339 | MELK |

TABLE 31

| UniGene Identifier | Gene Description |
|---|---|
| Hs.208124 | ESR1 |
| Hs.26225 | GABRP |
| Hs.504115 | TRIM29 |
| Hs.1594 | CENPA |
| Hs.184339 | MELK |
| Hs.592049 | PLK1 |
| Hs.370834 | ATAD2 |
| Hs.470477 | PTP4A2 |
| Hs.473583 | YBX1 |
| Hs.83758 | CKS2 |

TABLE 32
Genes associated with pre-menopause

| UniGene Identifier | Gene Description |
|---|---|
| Hs.208124 | ESR1 |
| Hs.26225 | GABRP |

TABLE 33
Genes associated with tumor grade

| UniGene Identifier | Gene Description |
|---|---|
| Hs.125867 | EVL |
| Hs.523468 | SCUBE2 |
| Hs.95612 | DSC2 |
| Hs.26010 | PFKP |
| Hs.437638 | XBP1 |
| Hs.444118 | MCM6 |
| Hs.469649 | BUB1 |
| Hs.591314 | GMPS |

TABLE 34
Genes associated with tumor grade 1

| UniGene Identifier | Gene Description |
|---|---|
| Hs.26010 | PFKP |
| Hs.437638 | XBP1 |
| Hs.444118 | MCM6 |
| Hs.469649 | BUB1 |

TABLE 35
Genes associated with tumor grade 3 or grade 4

| UniGene Identifier | Gene Description |
|---|---|
| Hs.523468 | SCUBE2 |
| Hs.95612 | DSC2 |
| Hs.591314 | GMPS |

TABLE 36

| | Median Relative | Range of |
|---|---|---|
| Gene ID | Expression* | Expression |
| EVL | 1.42 | 0.14-67.1 |
| NAT1 | 4.13 | 0.14-153.0 |
| ESR1 | 16.94 | 0-330.0 |
| GABRP | 4.55 | 0-1322.0 |
| ST8SIA1 | 0.65 | 0-7.9 |
| TBC1D9 | 0.97 | 0-63.4 |
| TRIM29 | 0.59 | 0-13.3 |
| SCUBE2 | 3.47 | 0-533 |
| IL6ST | 0.13 | 0-11.4 |
| RABEP1 | 0.72 | 0-10.0 |
| SLC39A6 | 0.64 | 0-31.4 |
| TPBG | 1.38 | 0.12-8.7 |
| TCEAL1 | 1.35 | 0-17.1 |
| DSC2 | 1.46 | 0.09-71.4 |
| FUT8 | 0.71 | 0-5.1 |
| CENPA | 0.19 | 0-1.8 |
| MELK | 0.18 | 0.02-1.8 |
| PFKP | 0.19 | 0.01-1.2 |
| PLK1 | 0.15 | 0.03-1.4 |
| ATAD2 | 0.45 | 0.09-4.0 |
| XBP1 | 6.84 | 0.39-40.5 |
| MCM6 | 0.18 | 0-2.8 |
| BUB1 | 0.10 | 0-1.0 |
| PTP4A2 | 0.61 | 0-6.0 |
| YBX1 | 0.27 | 0.01-1.4 |
| LRBA | 0.37 | 0.01-15.5 |
| GATA3 | 2.09 | 0.02-17.2 |
| CX3CL1 | 1.36 | 0.07-67.5 |
| MAPRE2 | 0.24 | 0-2.1 |
| GMPS | 0.29 | 0-4.1 |
| CKS2 | 0.16 | 0-2.4 |
| SLC43A3 | 0.26 | 0-1.4 |

*Relative to Universal Human Reference RNA (Stratagene)

While this invention has been particularly shown and described with references to example embodiments thereof, it will be understood by those skilled in the art that various changes in form and details may be made therein without departing from the scope of the invention encompassed by the appended claims.

What is claimed is:

1. A method for identifying a mammal having an increased likelihood of recurrence of breast cancer, comprising the step of identifying in a breast tissue sample of the mammal expression of at least two genes, wherein the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

2. The method of claim 1, wherein the expressed genes identified in the breast tissue sample consist of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

3. The method of claim 1, wherein the genes are selected from the group consisting of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1) and Hs.95612 (DSC2).

4. The method of claim 3, wherein the expressed genes identified in the breast tissue sample consist of Hs.125867 (EVL), Hs.591847 (NAT1), Hs.208124 (ESR1), Hs.26225 (GABRP), Hs.408614 (ST8SIA1), Hs.480819 (TBC1D9), Hs.504115 (TRIM29), Hs.523468 (SCUBE2), Hs.532082 (IL6ST), Hs.592121 (RABEP1), Hs.79136 (SLC39A6), Hs.82128 (TPBG), Hs.95243 (TCEAL1) and Hs.95612 (DSC2).

5. The method of claim 1, wherein the genes are selected from the group consisting of Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

6. The method of claim 5, wherein the expressed genes identified in the breast tissue sample consist of Hs.654961 (FUT8), Hs.1594 (CENPA), Hs.184339 (MELK), Hs.26010 (PFKP), Hs.592049 (PLK1), Hs.370834 (ATAD2), Hs.437638 (XBP1), Hs.444118 (MCM6), Hs.469649 (BUB1), Hs.470477 (PTP4A2), Hs.473583 (YBX1), Hs.480938 (LRBA), Hs.524134 (GATA3), Hs.531668 (CX3CL1), Hs.532824 (MAPRE2), Hs.591314 (GMPS), Hs.83758 (CKS2) and Hs.99962 (SLC43A3).

7. The method of claim 1, wherein the genes are selected from the group consisting of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

8. The method of claim 7, wherein the expressed genes identified in the breast tissue sample consist of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1), Hs.95612 (DSC2), Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

9. The method of claim 1, wherein the genes are selected from the group consisting of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1) and Hs.95612 (DSC2).

10. The method of claim 9, wherein the expressed genes identified in the breast tissue sample consist of Hs.208124 (ESR1), Hs.26225 (GABRP), Hs.480819 (TBC1D9), Hs.592121 (RABEP1), Hs.79136 (SLC39A6), Hs.82128 (TPBG), Hs.95243 (TCEAL1) and Hs.95612 (DSC2).

11. The method of claim 1, wherein the genes are selected from the group consisting of Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

12. The method of claim 11, wherein the expressed genes identified in the breast tissue sample consist of Hs.654961 (FUT8), Hs.1594 (CENPA), Hs.184339 (MELK), Hs.26010 (PFKP), Hs.592049 (PLK1), Hs.437638 (XBP1), Hs.444118 (MCM6), Hs.470477 (PTP4A2), Hs.473583 (YBX1), Hs.480938 (LRBA), Hs.524134 (GATA3), Hs.531668 (CX3CL1) and Hs.99962 (SLC43A3).

13. The method of claim 1, wherein the genes are selected from the group consisting of Hs.79136 (SLC39A6), Hs.82128 (TPBG), Hs.480819 (TBC1D9), Hs.592121 (RABEP1) and Hs.532082 (IL6ST).

14. The method of claim 13, wherein the expressed genes identified in the breast tissue sample consist of Hs.79136 (SLC39A6), Hs.82128 (TPBG), Hs.480819 (TBC1D9), Hs.592121 (RABEP1) and Hs.532082 (IL6ST) is identified in the breast tissue sample.

15. The method of claim 1, wherein the genes are selected from the group consisting of Hs.79136 (SLC39A6), Hs.82128 (TPBG), Hs.480819 (TBC1D9) and Hs.592121 (RABEP1).

16. The method of claim 15, wherein expression of Hs.79136 (SLC39A6), Hs.82128 (TPBG), Hs.480819 (TBC1D9) and Hs.592121 (RABEP1) is identified in the breast tissue sample.

17. The method of claim 1, wherein the genes are selected from the group consisting of Hs.79136 (SLC39A6), Hs.82128 (TPBG) and Hs.480819 (TBC1D9).

18. The method of claim 17, wherein expression of Hs.79136 (SLC39A6), Hs.82128 (TPBG) and Hs.480819 (TBC1D9) is identified in the breast tissue sample.
